(19) United States
(12) Patent Application Publication
(10) Pub. No.: US 2026/0249004 A1
(43) Pub. Date: Aug. 27, 2026

(54) Infusion Device

(71) Applicant: Michael Becker, Knittlingen (DE)

(72) Inventor: Michael Becker, Knittlingen (DE)

(21) Appl. No.: 18/578,318

(22) PCT Filed: Jul. 11, 2022
(86) PCT No.: PCT/EP2022/069330

§ 371 Date: Jan. 11, 2024

(30) Foreign Application Priority Data
Jul. 12, 2021 (DE) 10 2021 117 940.8

(51) Int. Cl. A61M 5/172; G16H 70/40

(52) U.S. Cl. CPC A61M 5/1723; A61M 2202/048; A61M 2205/502; A61M 2230/20; G16H 70/40

(57) ABSTRACT

In a procedure for determining a multimodal (patient) condition
a current (patient) state consisting of data from at least two unimodal state indicators Xₖ with k=1; . . . ; n and n≥2 is measured or calculated;
there is a historical (population) data set with data from at least two state indicators Xₖ and at least one reference indicator X₀;
there is a correlation between the at least two state indicators on the one hand and the at least one reference indicator on the other;
the at least two state indicators Xₖ and the at least one reference indicator X₀ span an orthogonal state space;
regression functions are calculated in the state space with the historical (population) data set, which contain at least one reference indicator X₀ as an independent variable;
and for a current (patient) state, at least one current, n-modal (n≥2) expectation value μ_{nM} of the currently unknown reference value is calculated on at least one reference axis X₀ of the state space using the regression functions.

### CROSS REFERENCE TO RELATED APPLICATIONS

This application is the U.S. national stage of International Application No. PCT/EP2022/069330, filed on 2022 Jul. 11. The international application claims the priority of DE 102021117940.8 filed on 2021 Jul. 12; all applications are incorporated by reference herein in their entirety.

### BACKGROUND

The invention relates to an infusion device for inducing and administering anaesthesia or sedation to a patient.

The aim is to significantly improve the accuracy of determining the depth of anaesthesia by broadening the database to include pharmacokinetic and pharmacodynamic model data (PkPd model) of the effective drug concentration and patient-specific physiological real-time data, and to maintain this accuracy over time or correct it as required.

Known methods such as target-controlled infusion (TCI) with an anaesthetic, for example propofol, are based on PkPd models, which, however, only inadequately estimate the patient-specific effective drug concentration. The measurement of patient-specific EEG data (electroencephalogram) and the index values derived from this as a measure of the depth of anaesthesia is also an established method. However, the correlation between these index values and the target concentration of the anaesthetic to be set is unsatisfactory due to high inter-individual variability.

Medical systems and methods are also known that consist of monitoring modules, control modules and drug delivery devices that use measured physiological data to regulate drug delivery.

U.S. Pat. No. 6,186,977 B1 describes a device system with at least one module for the intravenous administration of at least one drug and at least one module for monitoring physiological data, such as an electrocardiogram (ECG), blood pressure or respiratory data, which are related to the effect of the administered drug. The device system provides the user with information from multiple physiological data sources as a decision aid for maintaining or correcting the drug dose rate. However, the patent does not address how a specific decision aid could be derived from this independent monitoring data.

A TIVA TCI system (TIVA: total intravenous anaesthesia) is known from EP 1 278 564 B1, which contains several sensor modules for recording physiological data such as a blood pressure sensor and EEG sensors in order to monitor the anaesthetic state of a patient. In addition, the system contains a historical data set of a population that describes the effect relationship between a physiological datum, for example an EEG index datum, and a pharmaco-kinetic, -dynamically calculated drug concentration. This effect relationship of the population data is represented as a sigmoid function using a least square distance analysis. The drug delivery by the TIVA-TCI system is controlled with this population data-based sigmoid function, which only references sensor data of a single condition indicator. With respect to a current patient state, which is composed of the model-based calculated drug concentration and a current EEG index date, the population sigmoid function is shifted until the current patient state is in congruence with the sigmoid function. The shape of the function is not changed. The shifted sigmoid function defines a correlation between the currently measured EEG index value and the pharmacokinetically and dynamically calculated and adjusted drug concentration. A target value is defined on the EEG index scale, which generally does not initially correspond to the current EEG index date. However, the shifted sigmoid function provides the functional relationship as to how the current pharmacokinetically and dynamically calculated drug concentration must be changed in order to bring the current EEG index closer to the defined target value. The disadvantage of this method is that the pharmacokinetically and dynamically calculated drug concentration and the measured EEG index date have a large inter-individual variability with regard to the true, effective but unknown drug concentration. Nonlinear least square fit (LSD fit) algorithms for determining the sigmoid regression function are therefore not very robust, as there is generally a large, variable inter-individual variability of the data along both state axes. The sigmoid regression function to be calculated, which is used as the control function of the TCI method, is already dominated by only a few states with a randomly large distance to the regression function. Even orthogonal LSD methods are imprecise and not very robust with regard to the convergence behaviour when generating the regression function. No statement is made about the use of further state indicators.

EP 1 725 278 B1 describes a further development of EP 1 278 564 B1, in which a method is described for determining the sigmoid function as a regression function from a sequence of patient-specific states composed of pharmacokinetically and dynamically calculated drug concentrations and associated current physiological data, for example EEG index data. The sigmoid function is adapted to these current data using a least square distance fit procedure and used as the control function of a unimodal TCI method. Thus, in contrast to EP 1 278 564 B1, the shape of the sigmoid function is now determined by the sequence of patient-specific states. However, the scattering of the patient states around the regression function is disadvantageously large due to the inter-individual variability of the EEG index and the inter-individual variability of the pharmaco-kinetically, -dynamically calculated drug concentration, each of which is effective along its axis in a coordinate system. As already noted in EP 1 278 564 B1, this significantly disturbs the calculation of the regression function. Furthermore, it is known that the patient-individual correlation curves of EEG index over calculated drug concentration relatively often have flat plateaus and also areas with steep slopes. For example, the measured EEG index can fall steeply even at low calculated drug concentrations and asymptotically change to a horizontal plateau even at medium drug concentrations. However, the EEG index can also, depending on model-based, calculated drug concentrations, initially be flat with a high index value and only fall with relatively high calculated drug concentrations. The individualized regression curves are only suitable for achieving, maintaining or, if necessary, correcting a target value on the EEG index scale via a change in the model-based drug concentration with good convergence behaviour if the response profiles of the regression functions do not form flat plateaus over wide ranges.

The applicability of the methods described in EP 1 725 278 B1 is therefore limited. The methods described in EP 1 278 564 B1 and EP 1 725 278 B1 use only a single indicator to determine the depth of anaesthesia of a patient, which serves as a control variable for a unimodal TCI method. The methods are therefore not very robust and have inadequate convergence behaviour.

Also known are medical devices, systems and methods that transform physiological data from multiple monitoring devices that are indicative of anaesthetic depth or analgesia status to unnamed index scales in order to combine the index values with appropriate mathematical methods.

US 2006/0217614 A1 discloses a method for describing a patient condition, such as a pain condition, using data from several sensors. The different sensor data are each transformed to a nameless index scale and normalized. The transformation and normalization algorithm is based on historical data from a population. The transformed and normalized index data from different sensor sources are then used to calculate weighted, multimodal index values. For example, these are weighted mean values. However, no statement is made about the type of weighting. There is no discussion of how the normalized index values could be combined with a TCI.

U.S. Pat. No. 7,925,338 B2 describes a method, based on the method described above in US 2006/0217614 A1, in which physiological data are transformed and displayed as a normalized anaesthesia index and as a pain index. A patient condition is then graphically visualized in a two-dimensional plot, with one coordinate axis each being assigned to the pain index and the anaesthesia index.

WO2009/06346 discloses a method for mapping physiological data from several sensor modules, which are indicative of a patient's current pain condition, in a multidimensional state space. The state cloud of historical data of a population also exists in this state space. Using PCA (Principal Component Analysis) methods, the pain state is projected onto a common main axis of the state cloud with an index scale in order to reduce the pain state in the state space to one dimension. The current patient state projected onto the scaled main axis is then the multimodal datum of a current pain state. Inter-individual variabilities of the unimodal state indicators are methodically filtered out and remain unconsidered, especially for their relative weighting. However, non-linear data correlations are insufficiently taken into account when projecting onto a linear main axis.

US 2011/0137297 A1 also describes a system, consisting of several sensors, for recording physiological data that are monitored by a status monitor and that provides an output to control the dose rate of drug delivery devices. Essentially, the hardware architecture for anaesthesia and analgesia monitoring and drug delivery control is discussed. The methodology of data analysis and the generation of a control signal for the drug dose is not discussed.

WO 2012/171610 A1 discloses a method for combining physiological data from several monitoring sources in order to determine a multimodal patient state, whereby the number of data sources can be variably selected during monitoring.

Various established mathematical methods such as adaptive neuro-fuzzy logic, neural network methods, regression methods, vector machines or self-learning machines based on statistical methods are used to calculate this multimodal state. The adaptive fuzzy logic method is described in detail. TCI control of drug delivery using multimodal patient states is not discussed.

US 2016/0074582 discloses a method of controlling an infusion pump with a controller in order to achieve and maintain a defined target value. This is based on a multi-compartment model of the patient and the time-varying concentrations in the individual compartments are calculated pharmaco-kinetically and dynamically using a rate equation model. A drug concentration is assigned to the model compartment lung with a temporal sequence of measurements, for example the measurement of the drug concentration in the exhaled air. The PkPd model is adapted with regard to the measurements so that it can reproduce the time course of the measurement in the lung compartment. The personalized rate equation system is then used to determine a drug-dose profile in order to achieve and maintain the defined target concentration. The disadvantage of this is that the rate equation system is characterized by a large number of parameters according to the assumed number of compartments and can only be insufficiently personalized by measuring the drug concentration in a single accessible lung compartment.

US 2017/0181694 describes a system consisting of several sensors for recording physiological data, which together are indicative of a continuum of sedation depth. Their data is processed in a control module (transition monitor), which regulates the supply of medication for a patient. Different sensors are more or less suitable for determining the depth of sedation in different ranges of light, moderate or deep sedation. The sensor data are transformed to a nameless index scale for the depth of sedation. In a first step, the control module identifies the range of sedation depth and, in a subsequent step, weights a subset of suitable sensors, which then enable a more precise determination of the sedation depth in the identified range. Methods for processing the data in the control module to generate a control signal for drug delivery are not discussed.

Physiological data from different sensor sources that are indicative of the state of hypnosis or the state of analgesia generally have different inter-individual variabilities which are generally not constant over the clinically relevant range. The transformation and normalization of physiological data from several sensor modules to nameless index axes did not sufficiently take this into account. Weighting the different sensor data to calculate a multimodal index data is an option, but without further information it is also arbitrary. PCA algorithms separate the statistical noise, which is generated by the inter-individual variability of the data, from the information on the main axis. In particular, they do not weight the different quality of the sensor data. The PCA method also has weaknesses when non-linear correlations between the sensor data have to be taken into account. Adaptive fuzzy logic methods or neural networks for calculating multimodal patient conditions are self-learning systems that are able to take into account the different data quality or non-linearities. The parameters stored in the fuzzy logic methods and in the networks are determined in a training process that generally results in a large parameter set that has no physical or physiological significance and therefore remains opaque and is difficult to validate.

### SUMMARY

In a procedure for determining a multimodal (patient) condition
a current (patient) state consisting of data from at least two unimodal state indicators Xₖ with k=1; . . . ; n and n≥2 is measured or calculated;
there is a historical (population) data set with data from at least two state indicators Xₖ and at least one reference indicator X₀;
there is a correlation between the at least two state indicators on the one hand and the at least one reference indicator on the other;
the at least two state indicators Xₖ and the at least one reference indicator X₀ span an orthogonal state space;
regression functions are calculated in the state space with the historical (population) data set, which contain at least one reference indicator X₀ as an independent variable;
and for a current (patient) state, at least one current, n-modal (n≥2) expectation value μ_{nM} of the currently unknown reference value is calculated on at least one reference axis X₀ of the state space using the regression functions.

### DETAILED DESCRIPTION

The invention is based on the task of providing an infusion device with which a desired amount of an anaesthetic drug or pain drug can be administered to a patient in a substantially automated or automatic manner, as well as a corresponding method for operating the device. According to the invention, these tasks are solved by the features of the independent claims.

A device such as an infusion device is set up so that a control unit with a data memory and a data processing device is provided, as well as an input interface for receiving a plurality of unimodal patient data, and an output interface for controlling an infusion device for administering at least one anaesthetic drug to a patient, wherein the infusion device can calculate a quantity of the anaesthetic drug in accordance with the patient data received and the information stored in the data memory, such as in particular the historical data record, in order to be able to place the patient in a sufficiently deep anaesthesia, and the infusion device can be controlled via the output interface to deliver the corresponding quantity of the anaesthetic drug in terms of duration and quantity. It is understood, for example, that an infusion device is equipped with a corresponding pump in order to infuse a desired quantity of a liquid anaesthetic drug into a patient over a desired period of time. The anaesthetic drug can also be a gas. Other input signals include an EEG index, blood pressure, heart rate and other human factors known to the anaesthesiologist. Of course, the device can be pre-programmed for a desired depth profile of the anaesthesia. Warning or signalling devices can also be provided to emit an alarm signal if, for example, a breathing rate or a blood pressure undercut a threshold. The device can also be switched off if the anaesthetist so wishes.

The innovative system solution consists of a controller with a communication interface that exchanges data with established monitoring devices and infusion pumps. Current patient data xₖ are recorded from at least two unimodal status indicators Xₖ with k=1; . . . ; n and n≥2. These can be physiological patient data, for example blood pressure (MAP), or an EEG index derived from an electroencephalogram. However, a model-based, pharmaco-kinetically and dynamically calculated drug concentration in the blood or at the patient's effect site is also suitable as a unimodal condition indicator. The system solution uses a calibrated, historical population data set that maps the correlation of the unimodal condition indicators Xₖ as dependent variables on the one hand and at least one reference indicator X₀ as an independent variable on the other. Reference indicator data are, for example, the effective drug concentrations x₀ measured in blood samples. A reference indicator is a precisely measurable observable that defines a gold standard. The population data set calibrated in this way extends over a clinically relevant area and can be represented in an orthogonal state space whose coordinate axes are assigned to the state indicators and the reference indicators. The population data is mapped in regression functions and state density functions and stored in the device system as historical knowledge.

In application of the apparatus and the method, a current patient state, consisting of data of the at least two unimodal state indicators, is superimposed on the historical population data set in order to calculate therefrom at least one multimodal expectation value μ_{nM} of a reference indicator, for example the true but not measurable in real time and therefore unknown effective drug concentration x₀, on at least one reference axis of the state space. In a preferred method, probability densities are also assigned to the expectation values, whereby in particular the multimodal probability density is the personalized, adaptive confidence interval (CI) of the multimodal expectation value.

A particular embodiment of the method and the apparatus implements a robust, personalized, multimodal target-controlled infusion (PMM-TCI) algorithm, which is implemented as an “open-loop” or “closed-loop” control in order to iteratively approximate a current multimodal expectation value μ_{nM} to a defined target value c_{T} on the reference axis X₀. The relevant information is visualized in an intuitive display on a monitor of the apparatus.

Simulations based on clinical data provide multimodal expectation values with adaptive CI's that show an improved accuracy by a factor of two to three compared to classical unimodal TCI methods or unimodal monitoring devices.

It is understood that the features mentioned above and to be explained below can be used not only in the combination indicated in each case, but also in other combinations. The scope of the invention is defined only by the claims.

A few more advantages of the invention are mentioned below:
The use of multiple, unimodal state indicators broadens the database and thus fundamentally improves the accuracy of the calculated multimodal expectation value compared to xₖ—data of the unimodal state indicators and their unimodal expectation values.
The broader database increases the robustness of the control loop of a personalized multimodal TCI.
In addition to the state indicators Xₖ, a homogeneous population data set contains at least one reference indicator X₀, which is measured with high accuracy. From a mathematical point of view, this is a necessary prerequisite for least square distance fits (LSD fits) in order to calculate the correlation between the data of the state indicators Xₖ which might have a larger inter-individual variability and data of the reference indicator X₀ which have been measured with high accuracy and therefore with minimal statistical noise.
The reference indicator is used to calibrate the historical (population) data set. For this purpose, the inter-individual variability σₖ² (x₀) of the unimodal state indicators is determined with respect to arbitrary but fixed reference data x₀ in the preferred [X₀; Xₖ] plains of the state space. The inter-individual variabilities are generally variable over the clinically relevant range and can be represented as a regression function σₖ² (x₀).
The multimodal expectation values and multimodal probability densities are calculated from the xₖ data of the unimodal state indicators, which are ideally weighted in each section of the clinically relevant range with regard to their local inter-individual variability.
An inventive PMM-TCI algorithm is based on multimodal expectation values μ_{nM} with respect to true, effective, but at the time of the measurements unknown drug concentrations x₀, where the μ_{nM} are calculated from actual data xₖ of several unimodal state indicators Xₖ with k=1; . . . ; n. A regression function f₀ₖ (x₀) of population data in a [X₀; Xₖ] plane of the state space is used as a control function (“response curve”) to iteratively approximate the multimodal expectation value μ_{nM} with a control variable xₖ to a defined target value c_{T} on the reference axis X₀. In particular, the regression function f₀₁ (x₀) of the population is used as a control function, which describes the correlation between the pharmaco-kinetically-dynamically calculated drug concentration x₁ and the x₀ data of the reference indicator X₀ in order to iteratively calculate correction data {x_{1_j}|j=1; 2; . . . }. The correction datum x_{1_j} is transferred to an infusion pump, with which the infusion pump calculates and activates a suitable drug dose-time profile pharmacokinetically and dynamically in order to set and maintain a constant drug concentration x_{1_j} in the model. In each correction stage j, the xₖ data of the other status indicators Xₖ with k=2; . . . ; n are also recorded in order to generate a corrected multimodal expectation value μ_{nM_j}, which is then iteratively approximated to the target value c_{T} on the reference axis X₀.
In another preferred PMM-TCI algorithm, sequences of current patient data {x_{k_j}|k=1; . . . ; n; j=1; 2; . . . ; J} of the unimodal state indicators Xₖ are assigned pairwise to a sequence of current multimodal expectation values {μ_{nM_j}|j=1; 2; . . . ; J} and a sequence of current states {(μ_{nM_j}; x_{k_j})|k=1; . . . ; n; j=1; 2; . . . ; J} is generated in the [X₀; Xₖ] planes of a state space. At least one regression function f_{0k_pers_J}(x₀) with respect to this sequence of current states is used as a personalized multimodal control function to iteratively approximate the multimodal expectation value μ_{nM_j} to a defined target value c_{T} on the at least one reference axis X₀. In particular, the personalized control function f_{01_pers_J}(x₀) is used, which describes the patient-individual correlation between the pharmacokinetically-dynamically calculated drug concentration x₁ and the multimodal expectation value μ_{nM} on the reference axis X₀ in order to calculate in iteration step J+1 a correction datum x_{1_J+1}=f_{01_pers_J}(c_{T}). After transferring the corrected data x_{1_J+1} to a classic TCI module, which is implemented in an infusion pump for example, this generates a corrected drug dose profile in order to keep the drug concentration x_{1_J+1} constant in the model. In each correction stage J, the x_{k_J+1} data of the other state indicators Xₖ with k=2; . . . ; n are also recorded in order to generate a corrected multimodal expectation value μ_{nM_J+1}, which is iteratively approximated to a defined target value c_{T} on at least one reference axis X₀.
The multimodal control functions f₀ₖ (x₀) or f_{0k_pers_J}(x₀) result from the broad database of several state indicators, and thus enable robust, personalized, multimodal PMM-TCI, which are implemented as “open loop” or “closed loop” control circuits.
In particular, the control functions f₀₁ (x₀) and f_{0k_pers_J}(x₀), which represent the correlation between the multimodal expected values μ_{nM_j} and the pharmaco-kinetically-dynamically calculated drug concentrations x_{1_j} with j=1; . . . ; J, generally do not have flat intercepts, so that they are well suited for PMM TCI control circuits over the entire clinically relevant range.
The multimodal expectation values μ_{nM} retain their physical and physiological meaning as effective drug concentration in contrast to nameless multimodal index values on corresponding index axes. The multimodal expectation values μ_{nM} provide a bridge to existing empirical knowledge regarding effective drug concentration and depth of anaesthesia and classical TCI models;
The inventive method is based on a Bayesian statistical method for calculating multimodal expectation values and is a mathematically transparent, well-determined “top down” algorithm. In contrast, AI algorithms based on neural networks or fuzzy logic algorithms are “bottom up” algorithms that must first be trained and whose parameters generally no longer have any physical or physiological reference and lead to black box solutions: In contrast, the inventive method is therefore much easier to validate, particularly for clinical applications, due to its mathematical transparency.
The inventive method only needs to be validated once; the population can be gradually increased under defined inclusion criteria without having to revalidate the inventive method.
The inventive method is variable in terms of the number of unimodal state indicators used.

Further aspects of the invention are listed below for clarification.
The sequence of data {(μ_{nM_j}; x_{k_j})|k=1; . . . ; n; j=1; 2; . . . ; J} are weighted depending on their time of origin.
With the control functions f₀ₖ of the population or with the personalized multimodal control functions f_{0k_pers_j}, the x_{k_J+1}—data of the status indicators Xₖ are calculated prospectively in each iteration step j even before the correction date is activated in the infusion pump, which are to be expected when the target value c_{T} is reached, in order to prospectively indicate and avoid possible borderline violations of these data.

### BRIEF DESCRIPTION OF THE DRAWINGS

FIG. 1: Procedure for configuring the population. The pharmacokinetically and dynamically calculated target concentration of the anaesthetic drug is changed in stages. In each stage, all xₖ data of the state indicators Xₖ are measured and blood samples are taken to determine the effective drug concentration x₀, subsequently.

FIG. 2: Population data set in the [X₀; X₁] plane of the state space with pharmaco-kinetically-dynamically calculated drug concentration x₁ versus measured drug concentrations x₀ in blood samples.

FIG. 3: Population data set in the [X₀; X₂] plane of the state space with EEG index data (electroencephalogram) x₂ versus measured drug concentrations x₀ in blood samples.

FIG. 4: Population data set in the [X₀; X₃] plane of the state space with MAP data (mean arterial blood pressure) x₃ versus measured drug concentrations x₀ in blood samples.

FIG. 5: Fit of the frequency distribution (frequency fit) in the [X₀; X₁] plane in the analysis band around an x_{1M}=4.5 μg/mL date parallel to the reference axis X₀.

FIG. 6: Measured variance σ₂ₑ²(x₀) of the State indicator X₂ in the [X₀; X₂] plane of the state space.

FIG. 7: Convolution of three unimodal probability densities Pao (k=1; 2; 3) yield a multimodal probability density P_{3M}

FIG. 8: System for the application of a personalized multimodal target-controlled Infusion (PMM-TCI) comprises hardware and software modules.

FIG. 9: Flow chart of the PMM-TCI method A. Iterative approximation of multimodal expectation value μ_{3M} to the target c_{eT} with regression function f₀₁(x₀) of the population.

FIG. 10A-G: Process steps of PMM-TCI method A according flow chart from FIG. 9 in the [X₀; X₁] plane of the state space; Iterative approximation of multimodal expectation value μ_{3M} to the target c_{eT}.

FIG. 11: Flow chart describes the process of how the personalized control functions f_{0k_pers}(x₀) with k=1; . . . ; n are determined.

FIG. 12A-B: Process steps according flow chart from FIG. 11 in the [X₀; Xₖ] planes of the state space with k=1; 2.

FIG. 13: Flow chart of the personalized, multimodal PMM-TCI method B: The personalized regression function f_{0k_pers}(x₀) from FIG. 11 is iteratively improved by considering a temporal sequence of current patient states Sⱼ with j=1; . . . ; J: The regression function f_{01_pers_J}(x₀) yields a better convergence behaviour when approximating the multimodal expectation value μ_{3M} to the target c_{eT}.

FIG. 14A-D: Process steps of PMM-TCI method B according flow chart from FIG. 13 in the [X₀; X₁] plane of the state space; Iterative approximation of multimodal expectation value μ_{3M} to the target c_{eT}.

FIG. 15: Flow chart of time saving PMM-TCI method C: the personalized control function f_{01_pers_J}(x₀) is determined by a regression function that includes the zero-point patient state S₀ and a temporal sequence of current patient states Sⱼ with j=1; . . . ; J in the vicinity of the target value c_{eT}.

FIG. 16A-H: Process steps according flow chart from FIG. 15 in the [X₀; X₁] plane of the state space.

FIG. 17: PMM-TCI method D corrects the target concentration of the anaesthetic drug, for example Propofol c_{eT_Prop}, and also the target concentration of the analgesic drug, for example Remifentanil c_{eT_Remi}, in the [X_{0_Prop}; X_{0_Remi}] plane of the state space.

FIG. 18: Enlarged visualization of example from FIG. 17: ROC landmark for “Return of Consciousness” and a LOI landmark—“Loss of Consciousness” are risk minimizing information. Boundary limits shall not be exceeded or undershot in an ‘open loop’ or ‘closed loop’ control application.

### DETAILED DESCRIPTION OF THE PREFERRED EMBODIMENTS

There are n≥2 unimodal state indicators {Xₖ|k=1, . . . n}, whose data xₖ can be represented as a state vector in an n-dimensional state space:

$$\vec{S ( t )} = ( \begin{matrix}{x}_{1} ( t ) \\ {x}_{2} ( t ) \\ . \\ . \\ {x}_{n} ( t )\end{matrix} )$$

The unimodal state indicators Xₖ are, for example, physiological patient data such as blood pressure (mean arterial blood pressure MAP), heart rate (HR), heart rate variability (HRV), EEG index data (electroencephaloaram). They are easy to measure in routine clinical practice. A unimodal state indicator can also be a pharmacokinetically and dynamically calculated drug concentration.

In general, the state vector is variable over time. In stationary equilibrium, it is independent of time. In particular, different time constants of the state indicators are then not effective. The stationary n-dimensional state vector is then written:

$$\vec{S} = ( \begin{matrix}{x}_{1} \\ {x}_{2} \\ . \\ . \\ {x}_{n}\end{matrix} )$$

In addition, there is at least one reference indicator X₀ whose x₀ data correlate with the xₖ data of the unimodal state indicators Xₖ (k=1, . . . , n). If the unimodal state indicators are physiological data, there is a causal relationship. A reference indicator is the independent variable that has an effect on the dependent variable Xₖ. Reference indicators are used to calibrate the unimodal state indicators, whose absolute accuracies over a clinically relevant range are determined by a calibration. The reference indicators are precisely measurable observables and established gold standards. However, the x₀ data of the reference indicators are generally not determined in routine clinical practice due to the increased measurement effort involved. For example, these are drug concentrations of various drugs in blood samples from test subjects measured with high accuracy in the laboratory using an HPLC (High Precision Liquid Chromatograph). The state vector \vec{S} extended by a reference indicator X₀ in an (n+1)-dimensional state space is then written:

$$\vec{S} = ( \begin{matrix}{x}_{0} \\ {x}_{1} \\ . \\ . \\ {x}_{n}\end{matrix} )$$

A sequence of state vectors \vec{S}₁ with i=1, . . . , m with variable drug concentrations x_{0_i}, which are assigned to the reference indicator X₀ is written:

$${\vec{S}}_{i} = ( \begin{matrix}{x}_{0 \_ i} \\ {x}_{1 \_ i} \\ ⋮ \\ {x}_{n \_ i}\end{matrix} )$$

A sequence of state vectors \vec{S}₁ with i=1, . . . , m generates the population data set:

$$POP := [ \begin{matrix}{x}_{0 \_ 1} & … & {x}_{0 \_ m} \\ {x}_{0 \_ 1} & … & {x}_{0 \_ m} \\ ⋮ & & ⋮ \\ {x}_{n \_ 1} & … & {x}_{n \_ m}\end{matrix} ]$$

The unimodal state indicators Xₖ with k=1, . . . , n together with the reference indicator X₀ span an (n+1) dimensional state space, and the population POP can be represented as a state cloud of correlated indicator data in this space. Data analysis is preferably performed in the [X₀; Xₖ] planes of this state space.

In these planes, regression functions f₀ₖ (x₀) with k=1; . . . ; n, which are calculated using least square distance (LSD) fit methods, determine the functional relationship between the independent variable X₀ and the dependent variables Xₖ. The data points (x₀; xₖ) in the [X₀; Xₖ] planes scatter around the regression functions f₀ₖ(x₀). The scatter is described with indicator-specific variances σ²₀ (x₀) and σ²ₖ (x₀), which act parallel to the indicator axes X₀ and Xₖ.

The more precisely the data of the reference indicator can be determined, the more suitable it is as a reference and gold standard. The variances σₖ²(x₀) withk=1; . . . ; n are the indicator-specific, inter-individual variabilities of the indicators Xₖ in relation to an arbitrary but fixed reference date x₀ of a population. The scatter functions σₖ (x₀) are the absolute accuracies of the indicators Xₖ calibrated with the reference indicator X₀. The scatter σ₀ (x₀) of the reference indicator X₀ is the repeatability with which a datum x₀ can be measured. According to the invention, the reference indicator, which is established as the gold standard, is suitable for calibrating the state indicators Xₖ if the relative accuracy σ₀(x₀)/x₀ of the reference indicator is significantly better than the relative accuracy σₖ (x₀)/xₖ (x₀) of the state indicators Xₖ: The following relationship should apply:

$${σ}_{0} ( {x}_{0} ) / {x}_{0} < {σ}_{k} ( {x}_{0} ) / {x}_{k}$$

and preference should be given:

$${σ}_{0} ( {x}_{0} ) / {x}_{0} << {σ}_{k} ( {x}_{0} ) / {x}_{k} .$$

It is also particularly advantageous according to the invention if the reference indicator X₀ has an approximately constant absolute accuracy.

σ₀(x₀)≅const
over the relevant range. These conditions significantly facilitate the regression and variance analysis of the population data set.

The invention is explained in more detail below with reference to a drawing. The drawing contains a total of 18 figures. Quantities are given in the usual units.

In accordance with clinical data, a data set POP is configured in a simulation, which consists of n=3 condition indicators Xₖ with k=1; . . . ; 3 and a reference indicator X₀ as an example:
X₀: Reference indicator, is the propofol concentration measured in a blood sample
X₁: Pharmaco-kinetically-dynamically calculated drug concentration of the Anaesthetic drug propofol at effect-site (or in the blood plasma),
X₂: EEG index to determine the depth of anaesthesia,
X₃: MAP blood pressure (mean arterial pressure),

As a simulated example according clinical data, the population POP consists of 750 states and is mapped in a state space that is spanned by the three state indicators X₁, X₂, X₃ and a reference indicator X₀.

In the simulation, the reference datum x₀ of the reference indicator X₀ is the drug concentration in blood samples measured at steady-state equilibrium, which is determined with high accuracy using a HPLC spectrometer (Liquid High-Performance Chromatograph with the assumption: σ₀=0.1 μg/mL=const). The reference indicator is the independent variable, the state indicators Xₖ with k≠0 are the dependent variables.

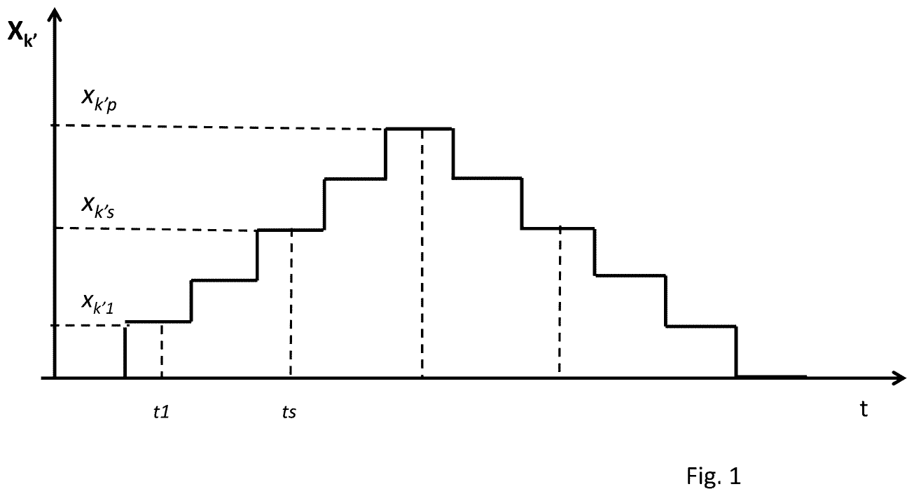

The configuration of a population requires a well-defined process flow and a well-defined structure in order to avoid systematic errors in the subsequent calculation of the regression functions and variances. FIG. 1 shows the procedure for configuring the population. For this purpose, the state indicator X₁, i.e., the pharmacokinetically and dynamically calculated target concentration of the anaesthetic drug, is preferably changed in stages as a manually determined variable with the infusion pump. In each stage, all xₖ data of the state indicators Xₖ are measured and blood samples are taken to determine the effective drug concentration x₀, subsequently.

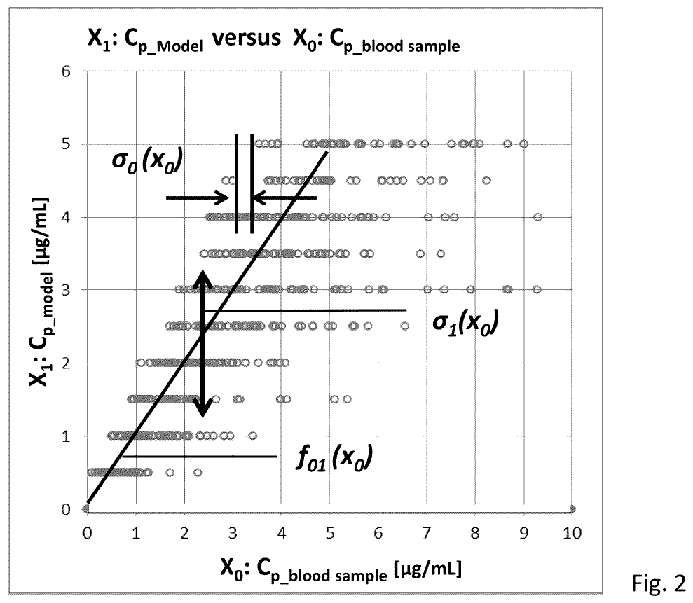

FIG. 2 shows a simulated population data set according to clinical data in the [X₀; X₁] plane of the state space with the manually determined variable X₁. The variable X₁ is changed in stages, whereby, for example, the same amount of data per stage is selected with the frequency F₁(x₁)=const with a limit value x_{1_max}=5 μg/mL, which avoids the risk of overdosing. For each state indicator Xₖ with k=1; . . . ; n, sequences of data {x_{k_i}|i=1; . . . ; m} are recorded simultaneously at each stage. Blood samples are also taken at each stage. The x_{0_i} data of the blood sample analysis are not known during the configuration process but are effective. They are subsequently assigned to the x_{k_i} data and form the population state vector \vec{S}₁=(x_{0_i}; x_{1_i}; . . . ; x_{n_i}). The variance σ₁² (x₀)≠const is effective parallel to the state indicator axis X₁, and it is truncated in the [X₀; X₁] plane due to the boundary condition x₁≤x_{1_max}=5 μg/mL for increasing x₀ data and therefore cannot be measured directly. Due to the boundary condition and a generally non-constant frequency profile F₁ (x₁)≠const along the X₁-axis, the regression function cannot be calculated using simple least square distance methods, which minimize the sum of the square distances parallel to the X₁-axis. The repeatability in the measurement of drug concentration in blood samples σ₀ is effective as the variance σ₀² parallel to the X₀ axis. It is assumed that the x₀ data can be measured very precisely in the laboratory using an HPLC measurement method, and the following applies: σ₀²≈const=0.1<<σ₁² (x₀).

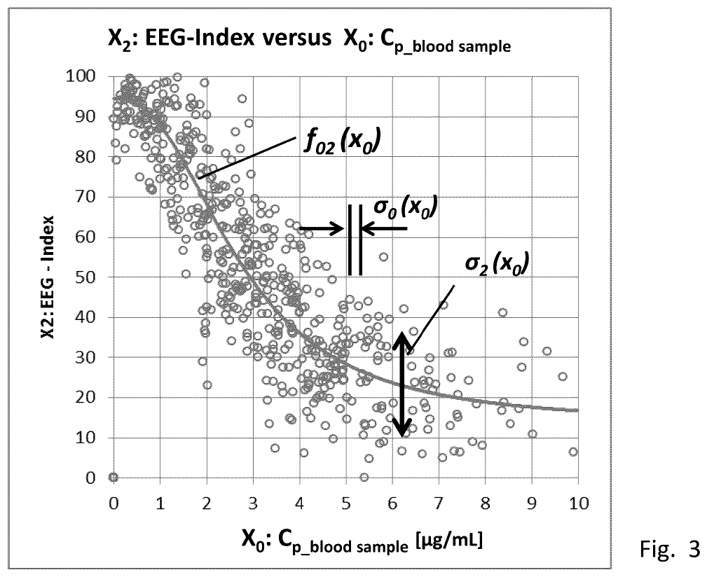

FIG. 3 shows the simulated population data set according to clinical data in the [X₀; X₂] plane. The state indicator X₂ represents an EEG index for the depth of anaesthesia. The x₂ data are recorded at the same time as the xₖ data of the other state indicators and the blood samples. The variance σ₂² (x₀) is effective parallel to the X₂ axis. The measured drug concentrations x₀ are not available at the time of the xₖ measurements. They are subsequently assigned to the xₖ data in all [X₀; Xₖ] planes, and they act as a true, unknown independent variable on all measured xₖ data with k=1; . . . n. Along the X₂—state indicator axis, no externally imposed frequency profile and no boundary conditions are effective, and the variance σ₂² (x₀) is not clipped.

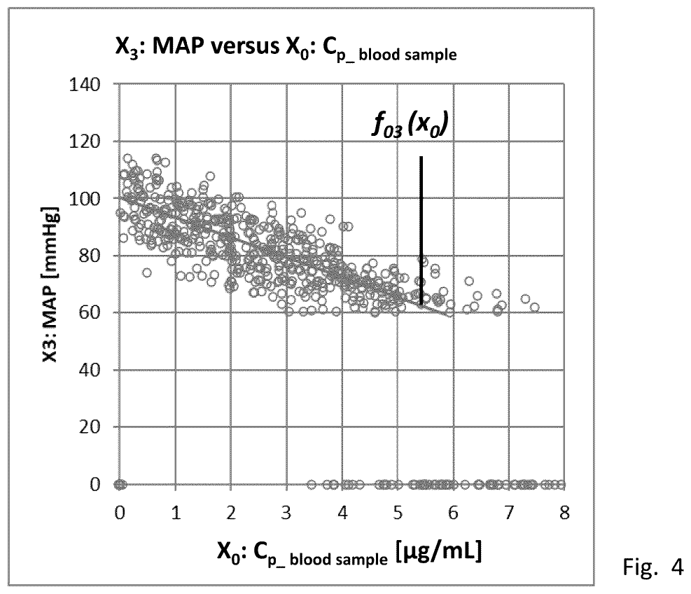

In accordance with clinical data, FIG. 4 shows an example of the population in the [X₀; X₃] plane of the state space with the state indicators X₃, i.e., the MAP (Mean Arterial Blood Pressure), which is also indicative of the depth of anaesthesia. A variance σ₃² (x₀)=const was assumed, exemplary. For X₃ a safety-relevant limit condition x_{3>60} mmHg applies.

In order to calculate the multimodal expectation values and probability densities, it is sufficient to perform the analyses in the preferred [X₀; Xₖ] planes of the state space, each of which contains the reference indicator X₀ as an independent variable. Narrow-band analysis bands are used for the variance and regression analysis of the population, i.e., narrow-band intervals in the preferred [X₀; Xₖ] planes of the state space. Each analysis band is aligned parallel to an indicator axis, and it is shifted over the clinically relevant range to perform data analysis. In [X₀; Xₖ] planes of the state space with boundary conditions along the Xₖ axis, the analysis bands are preferably oriented parallel to the X₀ reference axis and around an arbitrary but fixed x_{kM} datum of the indicator Xₖ because no boundary condition is effective along the reference axis.

Assuming that the state indicator X₁ was selected as a manually adjustable variable with a boundary condition x₁<x_{1_max} to configurate the population data set and a frequency F₁(x_{1M}) is given in the stage x₁ₘ, the state density in the analysis band parallel to the X₀ reference axis around the x₁ₘ datum in the [X; X₁] plane is:

$${D}_{01} ( {x}_{0} ; {x}_{1 M} ) = {F}_{1} ( {x}_{1 M} ) * \frac{\sqrt{{σ}^{2} ( {x}_{0} = 0 ) + {f}_{01}^{′2} ( {x}_{0} = 0 ) * {σ}_{0}^{2} ( {x}_{0} = 0 )}}{\sqrt{{σ}_{1}^{2} ( {x}_{0} ) + {f}_{01}^{′2} ( {x}_{0} ) * {σ}_{0}^{2} ( {g}_{01} ( {x}_{1 M} ) )}} * exp [ - \frac{1}{2} * ( \frac{{( {x}_{1 M} - {f}_{01} ( {x}_{0} ) )}^{2}}{{σ}_{1}^{2} ( {x}_{0} ) + {f}_{01}^{′2} ( {x}_{0} ) * {σ}_{0}^{2} ( {g}_{01} ( {x}_{1 M} ) )} ) ]$$

The state densities in the analysis bands of the other [X₀; Xₖ] planes with k=2, . . . , n at a distance of x_{kM} and parallel to the X₀ reference axis are written:

$${D}_{0 k} ( {x}_{0} ; {x}_{k M} ) = {F}_{0} ( {x}_{0} ) * \frac{\sqrt{{σ}_{k}^{2} ( {x}_{0} = 0 ) + {f}_{0 k}^{′2} ( x = 0 ) * {σ}^{2} ( x = 0 )}}{\sqrt{{σ}_{k}^{2} ( {x}_{0} ) + {f}_{0 k}^{′2} ( {x}_{0} ) * {σ}_{0}^{2} ( {g}_{0 k} ( {x}_{k M} ) )}} * exp [ - \frac{1}{2} * ( \frac{{( {x}_{k M} - {f}_{0 k} ( {x}_{0} ) )}^{2}}{{σ}_{k}^{2} ( {x}_{0} ) + {f}_{0 k}^{′2} ( {x}_{0} ) * {σ}_{0}^{2} ( {g}_{o k} ( {x}_{k M} ) )} ) ]$$

The sub-functions are:
Fₖ(xₖ): Frequency in analysis bands around xₖ and perpendicular to the indicator axis Xₖ;
f₀ₖ(x₀): The regression function in the [X₀; Xₖ] plane of the state space describes the correlation between the dependent variable Xₖ and the independent variable X₀;
g₀ₖ(xₖ)=f₀ₖ⁻¹ is the inverse of f₀ₖ;
f′₀ₖ(x₀)=df/dx₀: First derivative (slope) of the regression function at x₀.
f′²₀ₖ(x₀)*σ₀²(g₀ₖ (x_{kM}): Transformed variance term for the repeatability σ₀ of the reference indicator X₀, in the analysis band x_{kM} with a transformed effectiveness parallel to the Xₖ axis;
σₖ²(x₀): Variance term of the inter-individual variability of the state indicator Xₖ with respect to a reference value x₀ which is effective parallel to the Xₖ axis;
σₖ²(x₀)+f′²₀ₖ(x₀)*σ₀²(g₀ₖ (x_{kM}): The variance summation term adds the inter-individual variability σₖ²(x₀) of the state indicator Xₖ and the transformed variance of the reference indicator X₀. Due to the transformation both variance terms have an effectiveness parallel to the Xₖ axis and can be added together also under the general assumption σ²₀≠const.

The root terms in front of the exponential function normalize the function at the origin to 1.

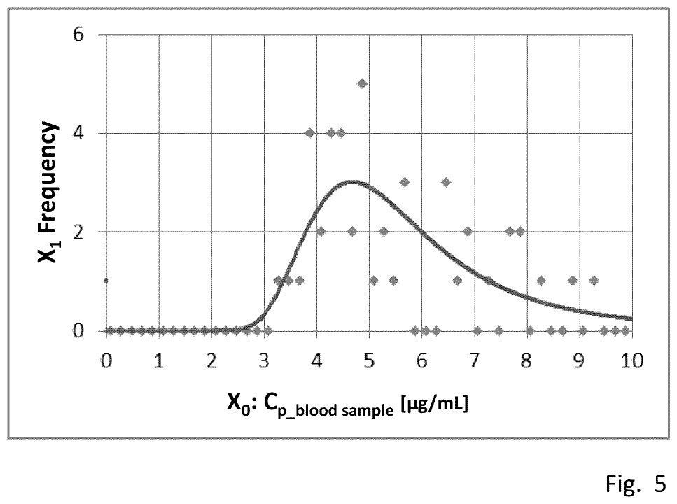

Since the manually selected variable X₁ is linked to a boundary condition x₁ₘₐₓ, the regression analysis in the [X₀; X₁] plane is preferably performed in analysis bands parallel to the reference axis X₀ to avoid disturbance. FIG. 5 shows a fit of the frequency distribution (frequency fit) in the analysis band around a datum x_{1M}=4.5 μg/mL parallel to the reference axis X₀. In each analysis band, the state density function D₀₁ of the frequency distribution is adjusted by an LSD fit. Polynomial approaches of the functions f₀₁ (x₀) and σ₁ (x₀) are used as fit variables. Averaging the polynomial coefficients in the respective analysis bands provides the regression function and variance function over the entire clinical area in the [X₀; X₁] plane.

In [X₀; Xₖ] planes of the state space, in which no boundary conditions are effective, classical least square distance methods are used to calculate the regression functions f₀ₖ ((x₀). In these planes, the variances σₖₑ² of the population are directly measured in analysis bands parallel to the respective Xₖ axis, because no disturbing boundary conditions are effective along these axes:

$${σ}_{ke}^{2} ( {x}_{0} ) := {{σ}_{k}^{2} ( x )}_{0} + {( {f}_{0}^{′} ( {x}_{0} ) * {σ}_{0} )}^{2}$$

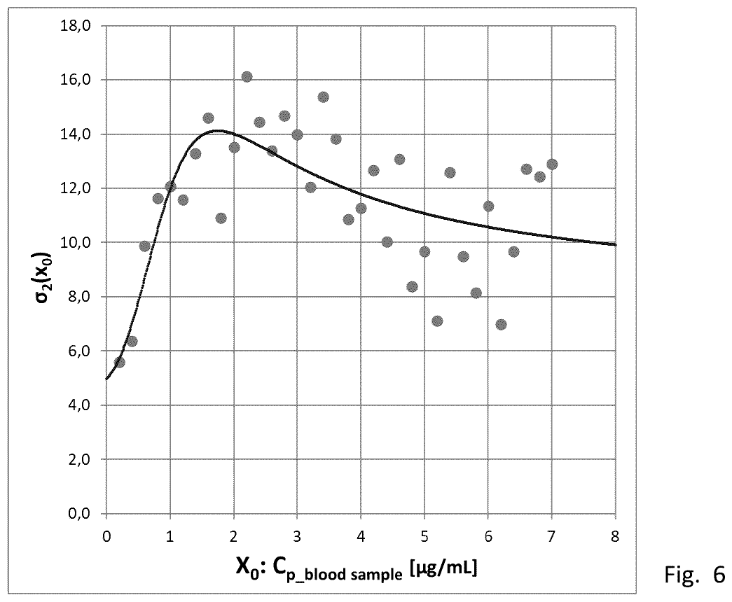

This is the measured inter-individual variability of the population for the indicator Xₖ in relation to an arbitrary but fixed reference value x₀. It contains the small non-constant variance term (f₀ₖ′(x₀)*σ₀)² of the finite but approximately constant repeatability σ₀ for measurements of the reference indicator. As an Example, the measured variance σ₂ₑ²(x₀) of the State indicator X₂ is shown in FIG. 6

The regression functions f₀ₖ (x₀) and variance functions σ²ₖₑ (x₀) determined from the analysis data contain the complete, in principle readable information of the population in parameterized form, which is used according to the invention in the application of the method and the apparatus for the calculation of expectation values and probability densities, which are immediately available without the need for re-assessed LSD fits.

In the routine clinical application of the inventive method and apparatus, current patient data xₖu of the state indicators Xₖ are measured or calculated pharmaco-kinetically and dynamically. However, no current reference data x_{0M} of the reference indicator X₀ are measured, as the effort involved is too great, and these x_{0M} data are not accessible in real time, for example because they are the result of a laboratory analysis. The current xₖM patient data with k=1; . . . ; n of the state indicators without the x_{0M} data of a blood sample analysis are therefore superimposed on the historical population data set, and the expectation values and the probability densities of the true but unknown effective drug concentration are calculated instead of measured x_{0M} data and projected on X₀. According to the invention, the expectation values μₖ, in particular the multimodal expectation values μ_{nM}, replace the x_{0M} data from blood sample analyses that are not available in routine use.

From the state density functions D₀ₖ in the [X₀; Xₖ] planes, the probability densities P₀ₖ (x₀; x_{0M}) of the unknown, effective drug concentration x₀ for a current, measured or model-based calculated patient date x_{kM} parallel to the reference axis X₀ are determined by renormalization with the normalization constant Nₖ(x_{kM}):

$${P}_{0 k} ( {x}_{0} ; {x}_{kM} ) = N k * \frac{{σ}_{k e} ( {x}_{0} = 0 )}{{σ}_{k e} ( {x}_{0} )} * exp [ - \frac{1}{2} * ( \frac{{( {x}_{kM} - {f}_{0 k} ( {x}_{0} ) )}^{2}}{{σ}_{k e}^{2} ( {x}_{0} )} ) ]$$

The function P₀ₖ (x₀; x_{kM}) is the probability that, knowing the current measured value x_{kM}, the variable x₀ on the reference axis X₀ is identical to the true but unknown effective drug concentration x_{0M}.

The multimodal probability density P_{nM} (x₀; x_{1M}; . . . x_{nM}) is the convolution of the indicator-specific, unimodal probability densities P₀ₖ (x₀; x_{kM}) along the common reference axis X₀, which is also the result axis. The function P_{nM} (x₀; x_{1M}; . . . ; x_{nM}) is the probability that, knowing all currently measured or calculated xₖ, values with k=1, . . . , n, the variable x₀ is identical to the unknown but true reference indicator value x_{0M}. According to the invention, the reference indicator axis becomes the result axis.

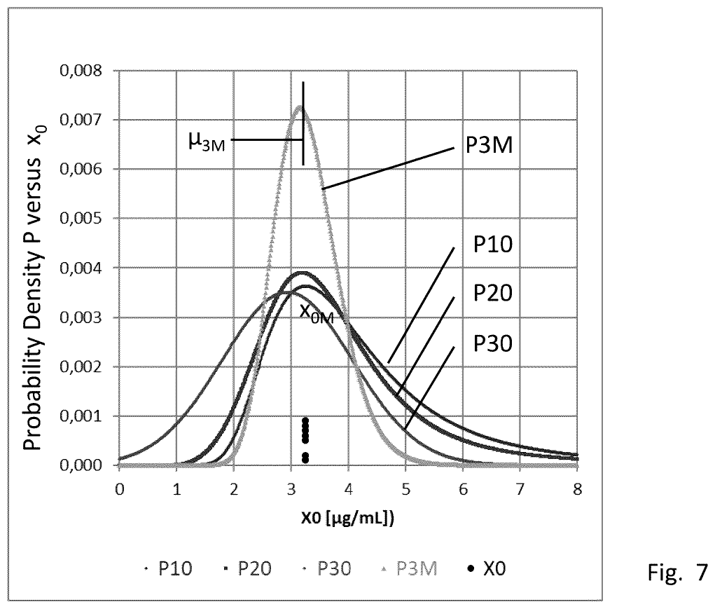

This process is shown as an example in FIG. 7. The effective drug concentration x_{0M} assumed in the simulation is shown as a vertical bar. The probability densities Poi with respect to the pharmaco-kinetically-dynamically calculated effect site concentration x_{1M} for the intravenously administered anaesthetic drug propofol and the probability density P₀₂ with respect to the measured EEG index x_{2M} are asymmetric functions parallel to the reference axis X₀. The asymmetry is caused by the variable variances σ²₁ (x₀) and σ²₂ (x₀) and by the non-linearity of the regression function f₀₂ (x₀). The probability densities P₀₃ for the blood pressure (MAP: mean arterial pressure) is a symmetrical function with respect to a maximum value, because σ²₃ (x₀)=const was used in the simulation. The probability densities P₀₁, P₀₂, P₀₃ are normalized to the surface normal 1, and their maxima are grouped around the effective but unknown reference value x_{0M} in the real application. The triple-modal probability density P_{3M} is also an asymmetric function with regard to its maximum value and is normalized to the area normal 1. It is noteworthy that in the simulation the maximum of P_{3M} is very close to the effective but in the real application off course unknown blood concentrations x_{0M}=3.25 μg/m. In addition, the profile of the multimodal probability density P_{3M}(x₀) is considerably narrower than the indicator-specific, unimodal probability densities P₀ₖ. This demonstrates the inventive advantage that multimodal expectation values and multimodal probability densities estimate the effective but unknown true drug concentration in real time much more accurately than is possible with unimodal expectation values and unimodal probability densities.

The multimodal expectation value of the effective but unknown drug concentration at the time of measurement of a current patient condition can be determined in various ways.

Knowing the regression function f₀ₖ (x₀), the unimodal expectation value μₖ (x_{kM}) with respect to a currently measured or calculated date x_{kM} of the unimodal state indicator Xₖ can be determined by the inverse of f₀ₖ⁻¹ if it exists:

$${μ}_{k} ( {x}_{kM} ) = {f}_{0 k}^{- 1} ( {x}_{kM} )$$

The mean value of the indicator-specific expectation values μₖ (x_{kM}) with k=1; . . . ; n is then the multimodal expectation value μ_{M} (M: Mean):

$${μ}_{M} = \frac{1}{n} * ( {\sum }_{k = 1}^{n} {μ}_{0 k} )$$

The disadvantage of this is that the indicator-specific, unimodal expectation values μₖ are equally weighted. However, the probability densities P₀ₖ, have different profile widths, which are caused by the different inter-individual variabilities of the indicators and/or any non-linearities of the regression functions f₀ₖ. Simple averaging does not take this information into account and therefore leads to systematic errors.

It is therefore advantageous to use the indicator-specific variances σ²ₖ(x₀) as weighting factors for calculating the multimodal expectation value μ_{WM} (WM: Weighted Mean):

$${μ}_{WM} = \frac{1}{{\sum }_{k = 1}^{n} \frac{1}{{σ}_{k}^{2} ( {μ}_{k} )}} * ( {\sum }_{k = 1}^{n} \frac{{μ}_{k}}{{σ}_{k}^{2} ( {μ}_{k} )} )$$

The disadvantage of this is that the asymmetries of the probability densities P₀ₖ, which are caused by the non-constant functions σ²ₖ(x₀) and non-linearities of the regression functions, are not taken into account.

The classic definition of the expectation value with respect to an x_{kM}—date of the state indicator Xₖ is

$${μ}_{k} ( {x}_{kM} ) = \int {x}_{0} * {P}_{0 k} ( {x}_{0} ; {x}_{k M} ) * d {x}_{0}$$

The calculation of the expectation value takes into account the asymmetry caused by non-constant variability σ²ₖ (x₀) and by non-linearities of the regression functions f₀ₖ, which is mapped in the probability density P₀ₖ. This means that the complete information content of the population is used to calculate the expectation values. The multimodal expectation value μ_{nM} (nM: n-fold modal) with regard to the current patient data {x_{kM}|k=1; . . . ; n} of the unimodal state indicators Xₖ is then written:

$${μ}_{nM} ( {x}_{1 M} ; … ; {x}_{nM} ) = \int {x}_{0} * {P}_{nM} ( {x}_{0} ; {x}_{1 M} ; … . ; {x}_{nM} ) * d {x}_{0}$$

The expectation value μ_{nM} is the projection of the “centre of gravity” of the asymmetric multimodal probability density P_{nM} onto the reference axis. This is an average value with respect to repeated measurements on a cohort with approximately the same effective drug concentration x_{0M}.

However, the expectation value μ_{MnM} (MnM: maximum of the n-modal probability density) for a single measurement of a current patient condition (x_{1M}; x_{2M}; . . . ; x_{nM}) is determined by the maximum of the probability density:

$${μ}_{MnM} = max ( {P}_{nM} ( {x}_{1 M} ; … ; {x}_{nM} ) )$$

The calculation of the expectation values according to methods 3 and 4 provides only insignificant differences in the example, which is based on clinical data.

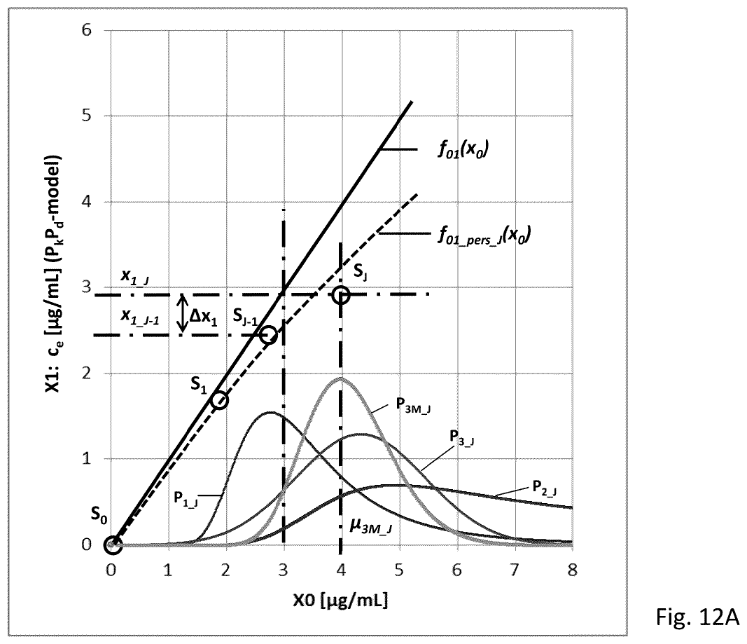

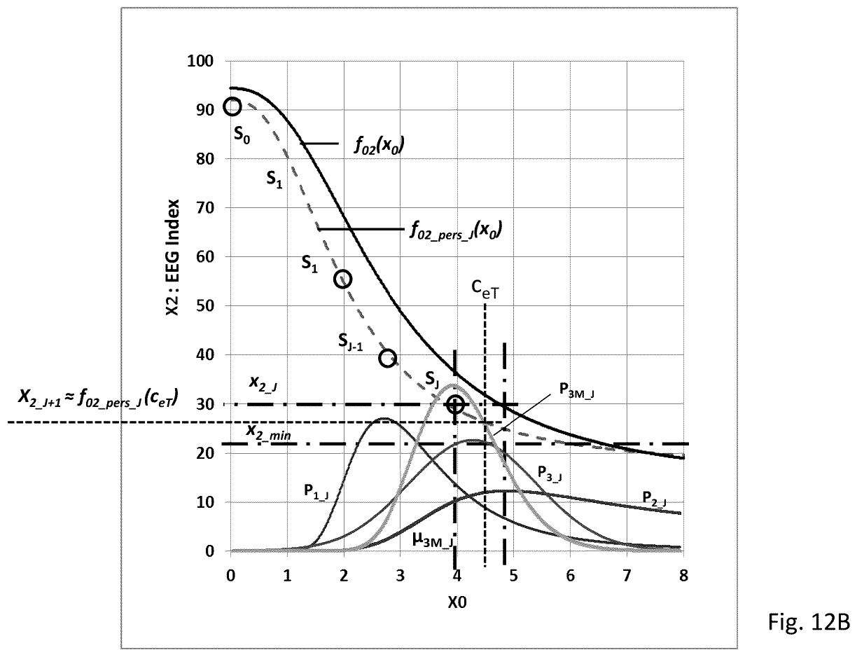

The unimodal probability densities from FIG. 7 are unimodal confidence intervals for the expectation values μₖ with respect to current unimodal measured values x_{kM} with k=1; . . . ; 3. The confidence intervals are variable depending on the currently measured x_{kM} data of the state indicators. The triple-modal probability density P_{3M} (x₀; x_{1M}; x_{2M}; x_{3M}) from FIG. 12 is a function of the current patient-specific measurements x_{kM} with k=1; . . . ; n, which are superimposed on the population. The width of the multimodal probability density P_{3M} is a personalized dynamically adaptive confidence interval with respect to the expectation value μ_{3M}. It is of inventive advantage, in addition to displaying the multimodal expectation values, also to display the indicator-specific unimodal and/or the multimodal probability density and/or the confidence intervals on a monitor of the apparatus in order to determine and visualize the quality of the current measurement in relation to historical data.

The expectation values and assigned confidence intervals must be confirmed in a validation process. Based on a historical population data set, it must be shown how well the multimodal probability densities P_{nM} include the true, effective drug concentration x_{0M} determined in a laboratory analysis. In the simulation, more than 75% of the true reference values x_{0M} lie within the simple FWHM width of the triple-modal probability density P_{3M}.

In the following, the “Open Loop” and “Closed Loop” methods and the apparatus for a personalized multimodal TCI (PMM-TCI) are described, which uses the broad database of several state indicators Xₖ with k=1; . . . ; n to iteratively approximate and obtain multimodal expectation values μ_{nM} of true, effective but unknown drug concentrations x₀ to a target value c_{T} on the reference axis X₀. Advantages and differences of the personalized, multimodal PMM-TCI methods to established, classical TCI methods are:

The PMM-TCI method uses the multimodal expectation value μ_{nM} of the effective but unknown drug concentration x₀, which is essentially determined by current physiological patient data. The multimodal expectation value μ_{nM} is approximated with an “open-loop” or “closed-loop” control of a defined target concentration c_{T} on the reference axis X₀, and corrected if necessary. The use of current physiological patient data to calculate a multimodal expectation value of an effective drug concentration personalizes the PMM-TCI method. In a classic TCI method, the drug concentration is only calculated pharmacokinetically and dynamically based on the model.

The multimodal probability density has a significantly lower standard deviation than a unimodal probability density. A defined target concentration can be set and obtained with the PMM-TCI method using the multimodal expectation value of the effective drug concentration with much greater accuracy than is possible with classical TCI methods;

The convergence behaviour and robustness of a personalized, multimodal PMM-TCI method for goal achievement and goal maintenance are significantly better compared to unimodal TCI-methods, since the PMM-TCI uses the broader database of several state indicators, including physiological patient data.

The patient's reactions caused by stress induction, for example an increased heart rate during a surgical procedure, are regulated to a stress-free level with the PMM-TCI. For this purpose, the target concentration of the anaesthetic drug, for example propofol, or the analgesic, for example remifentanil, is increased and the multimodal or unimodal expectation values of the effective but unknown drug concentrations x_{0_Prop} or x_{0_Remi} are approximated to the corrected target values c_{eT_Prop} and c_{eT_Remi} in a controlled manner on the respective drug-specific reference axis X_{0_Prop} or X_{0_Remi}. By specifying a fixed, preset target concentration of the analgesic c_{eT_Remi}, the target concentration of Propofol c_{eT_Prop} is preferably changed in order to minimize the patient's stress-induced reactions. If necessary, for example if the propofol target concentration c_{eT_Prop} is already very high, or if limit conditions would be violated, the preset target concentration of the analgesic is also corrected according to the invention in order to minimize the patient's reaction to stress-induced disturbance.

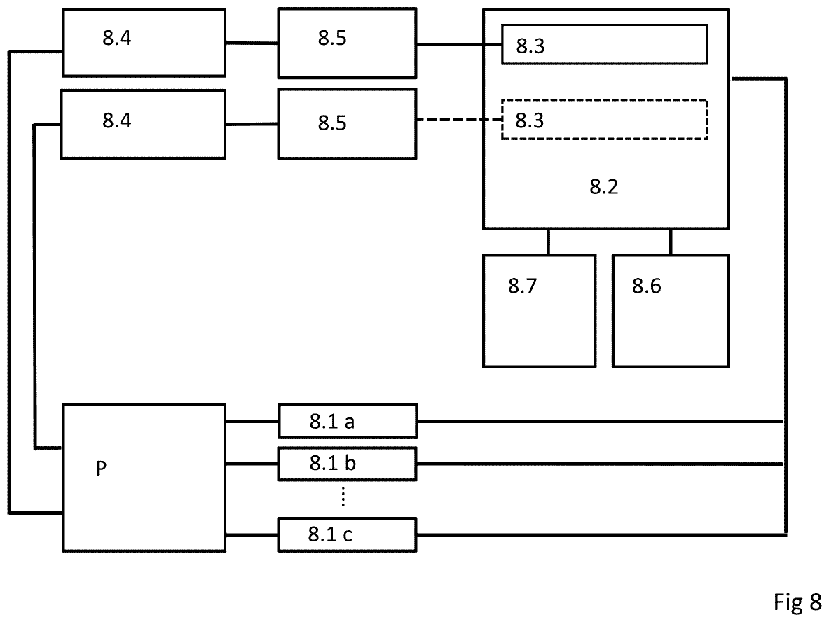

The inventive system for the application of a PMM-TCI comprises hardware and software modules as shown in FIG. 8. At least one module for the infusion 8.4 (infusion module hypnosis and infusion module analgesia) of medication is connected to the patient P. At least one sensor 8.1 records physiological patient data indicative of the drug effect. These sensor-based state indicators are, for example, the EEG index X₂, or a mean arterial blood pressure (MAP) X₃. There are also sensors that record the patient's reactions to stress induction. These are, for example, hemodynamic data such as heart rate (HR) or heart rate variability (HRV). The xₖ data of the state indicators Xₖ with k=1; . . . ; n are read by the controller 8.2 and further processed by the PMM-TCI software modules 8.3 (PMM-TCI Hypnosis and PMM-TCI Analgesia). The controller is connected on the one hand to a data input and output interface 8.7 and on the other hand to at least one module for drug application. This is, for example, an infusion pump 8.4. The controller is also connected to a memory module 8.6, in which a calibrated population data set with the historical knowledge of the application is stored. The infusion of hypnotics or analgesics with a defined dose-time profile is controlled with the classic PₖP_{d}-TCI software modules 8.5 (PₖP_{d}-TCI module hypnosis and PₖP_{d}-TCI module analgesia). The PₖP_{d}-TCI software modules 8.5 are established pharmaco-kinetic-dynamic models, for example the Marsh model or the Minto model. They are integrated in the infusion pumps or in the controller.

The controller monitors the infusion pumps and regulates the drug supply in an “open-loop” or “closed-loop” operating mode.

The personalized, multimodal TCI software modules (PMM-TCI modules) 8.3 for anaesthesia (optionally also for analgesia) of the controller work with data of the unimodal state indicators Xₖ with k=1; . . . ; n. These are pharmaco-kinetically and -dynamically calculated drug concentrations for an anaesthetic, for example propofol X_{1_Prop}, and/or for an analgesic, for example remifentanil X_{1_Remi}, and the physiological patient data of the sensor modules X₂; . . . ; Xₙ. The current, measured or pharmacokinetically-dynamically calculated patient data x_{kM} of the unimodal state indicators Xₖ are superimposed on the population data set, and at least one drug-specific PMM-TCI module 8.3 of the controller calculates a personalized, multimodal expectation value μ_{nM} on at least one reference axis X₀ and compares it with at least one target concentration c_{eT}, which is also defined on the respective reference axis and is inscribed via the user interface. At least one PMM-TCI module 8.3 of the controller calculates a correction value of the model-based drug concentration x₁ depending on the target deviation of the multimodal expectation value μ_{nM} from the defined target value c_{eT} and transmits it to the classical PₖP_{d}-TCI software module 8.5. the PₖP_{d}-TCI software module corrects the drug-dose-time profile of the infusion pump 8.4 with the transferred correction datum x₁ in order to keep the x₁ datum constant in the model. The correction datum x₁ is a dependent, iteratively adjusted control variable in order to approximate the multimodal expectation value μ_{nM} to the target value c_{eT} on the reference axis X₀. The x₁ correction date should not be confused with the target value c_{eT} defined on the reference axis X₀.

The user interface 8.7 is used to write data from the user to the controller in order to configure the system and to visualize data from the controller. In “Open-Loop” mode, the user is shown suggestions for correcting the medication. They can confirm the suggestions or change them manually if necessary. The modules of the system can be individual devices or they can be set up in different integrated configurations.

In the following, the inventive personalized, multimodal PMM-TCI methods are explained in detail using the example of a TIVA-TCI for propofol. They are also suitable for sedation with a low-dose anaesthetic.

The inventive PMM-TCI methods start with the induction phase, in which the patient's anaesthesia is induced and the system is calibrated. The target concentrations in the blood plasma or on effect site for propofol c_{eT_Prop} and for remifentanil c_{eT_Remi} are defined and set one after the other. In the following, a personalized, multimodal Propofol-PMM-TCI is used together with a classical Remi-TCI which is based e.g., on the Minto model. A Remi-PMM-TCI could also be realized for the analgesic, but a detailed description is not provided. Finally, depending on the clinical indication, a muscle relaxant is administered prior to intubation in preparation for mechanical ventilation.

Various inventive personalized, multimodal PMM-TCI methods A, B, C, D are explained in detail below using the example of total intravenous anaesthesia (TIVA) with propofol.

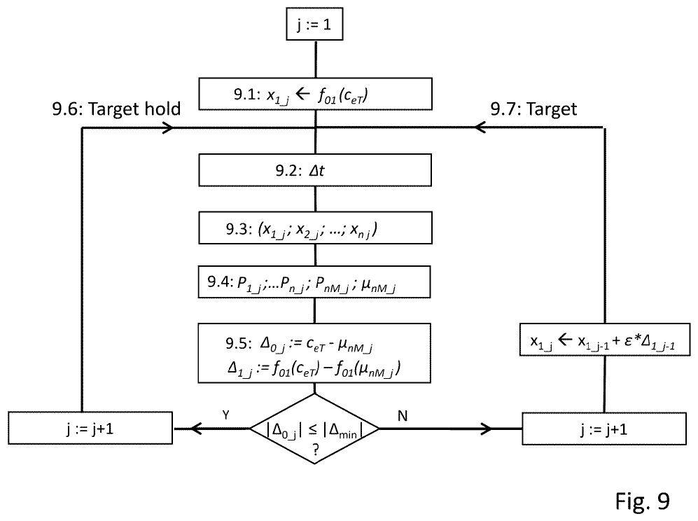

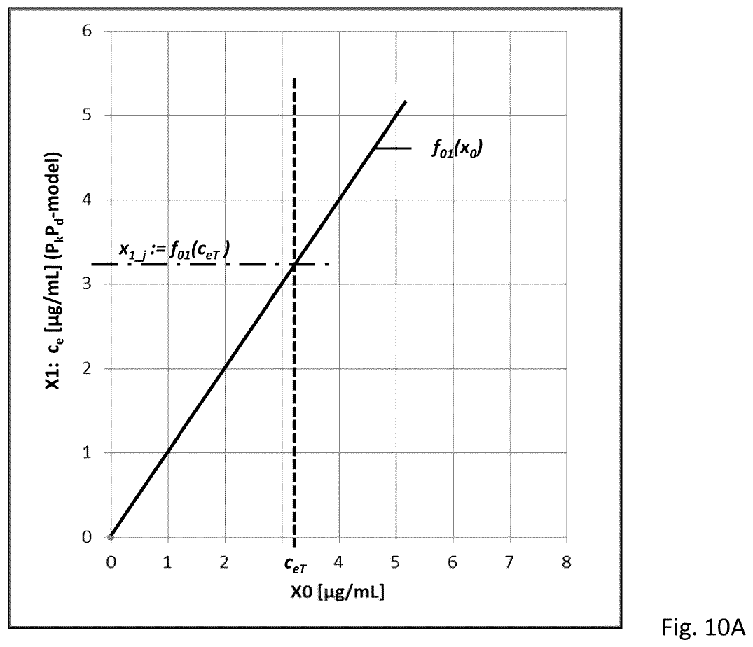

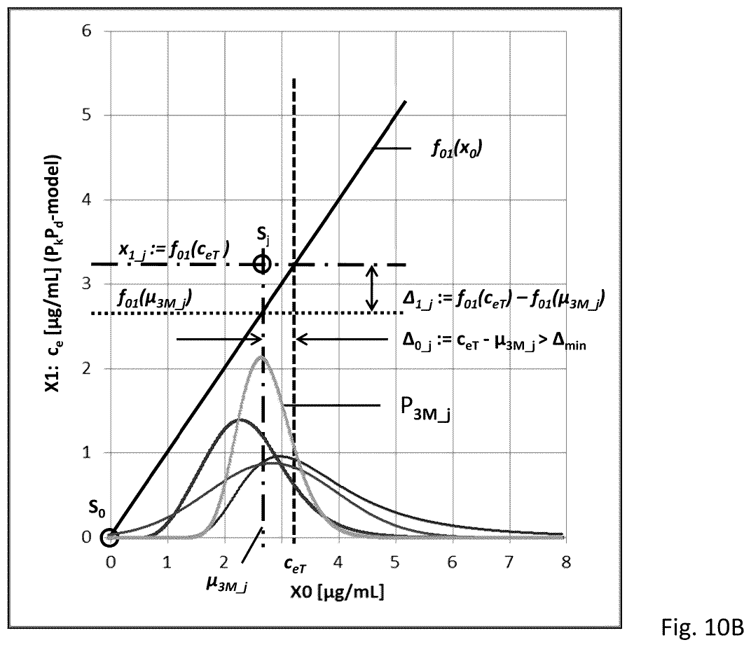

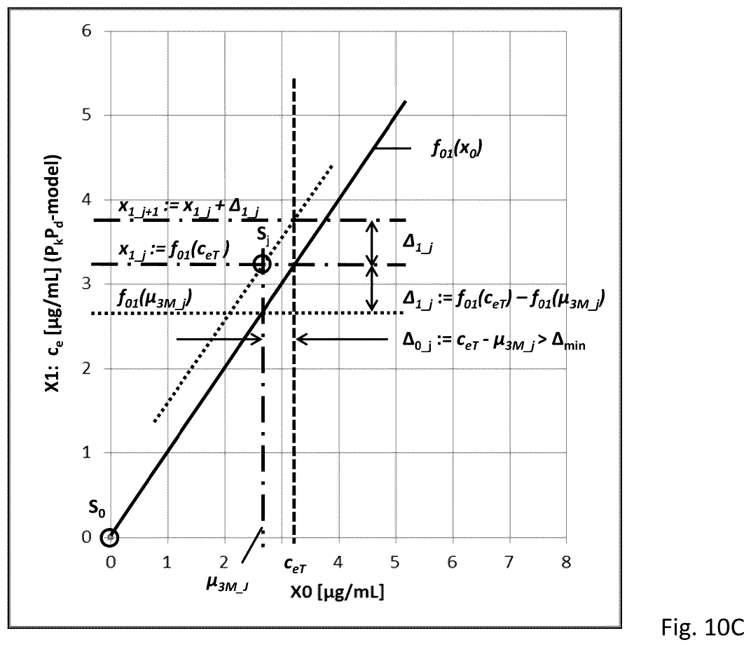

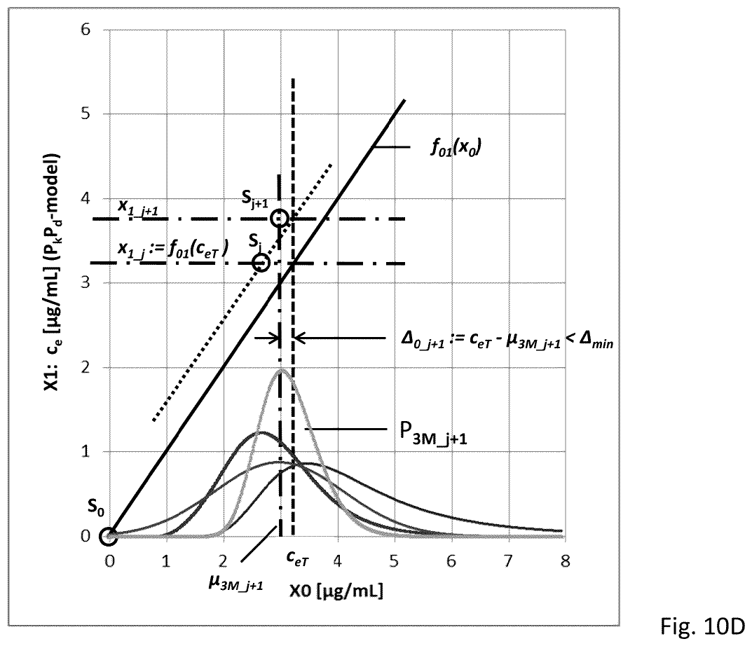

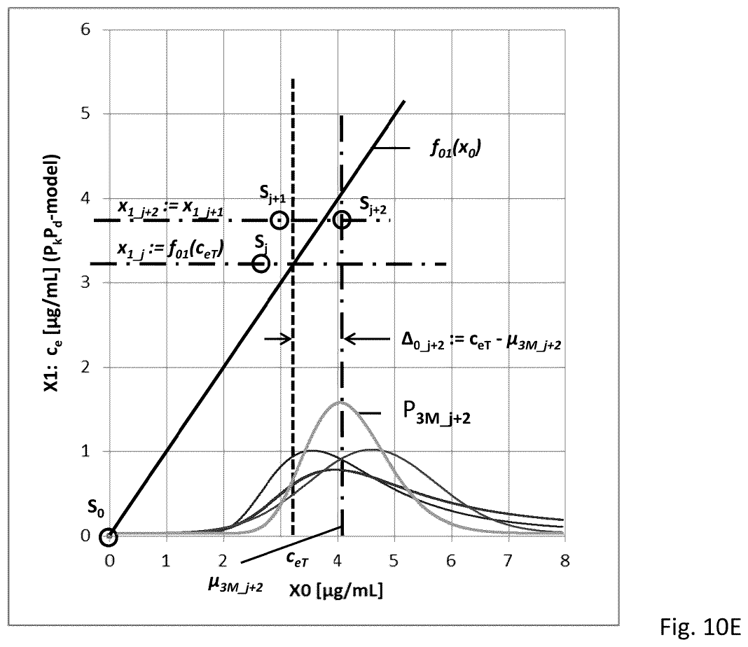

The inventive, personalized, multimodal PMM-TCI method A uses the regression function f₀₁ of the population POP as a control curve to approximate the multimodal expectation value μ_{nM_Prop} to the target value c_{eT_Prop} in an iterative process. A stress-induced disturbance is initially excluded. In the induction phase, the target value _{ceT_Remi} is defined and set. For example, a Minto model, which is a classic Remi-TCI algorithm, is used for this purpose. A target value c_{eT_Prop} is then defined and set on the reference axis X_{0_Prop}. The detailed description of the inventive PMM-TCI method A is explained in the flow chart of FIG. 9 and in the sequence of FIGS. 10A-G visualizes the process steps in the [X₀; X₁] plane of the state space.

FIG. 9 shows a TCI in mode A. The regression function f₀₁ (c_{eT}) of the population data in the [X₀; X₁] plane of the state space is used as a control function to determine the first dependent x_{1_j} datum of the indicator X₁ in the first iteration level j=1 (9.1): x_{1_j}:=f₀₁ (c_{eT}). FIG. 10A visualizes this step. The x_{1_j} datum is transferred to a classical (!) PₖP_{d}-TCI module 8.5, for example a Marsh or Schnider model, with which the infusion pump 8.4 calculates and activates a suitable drug dose-time profile pharmacokinetically and dynamically in order to constantly set and maintain the transferred drug concentration x_{1_j} in the model. The x_{1_j} datum is a dependent control variable and should not be confused with the target value c_{eT_Prop} defined on the reference axis. After an interval time 9.2 Δt≈3-4 minutes, a stationary equilibrium of the drug concentration in the patient is reached, and the other dependent data 9.3 {x_{k_j}|k=2; . . . ; n} of the state indicators Xₖ are then also measured with the sensors 8.1. The superimposition of the current patient data 9.3 {x_{k_j}|k=1; . . . ; n} with the population provides the associated unimodal expectation values μ_{k_j} and the probability densities P_{k_j} with k=1; . . . ; n. The multimodal probability density Pnu_Jand the multimodal expectation value μ_{nM_j} of iteration level j are then calculated from this in 9.4.

FIG. 10B shows the projection of this currently measured patient state \vec{S}ⱼ=(μ_{nM_j}; x_{1S_j}; x_{2_j}; . . . ; x_{n_j}) in the [X₀; X₁] plane of the state space for the number n=3 of state indicators. In this, the true but unknown drug concentration x_{0_j} at the time of measurement is substituted by the multimodal expectation value μ_{nM_j}. The deviation Δ_{0_j}=c_{eT}−μ_{nM_j} of the multimodal expectation value μ_{nM_j} from the defined target value c_{eT} on the reference axis X₀ is then determined (9.5). In the example, it is initially greater than the defined limit condition Δₘᵢₙ. In further iteration steps j=2; 3; . . . ; J, the dependent variable x_{1_j} is changed iteratively until the deviation finally fulfils the limit condition Δ_{0_j}≤Δₘᵢₙ for target achievement. In the example shown in FIG. 10B, the effective concentration of the drug must be increased in order to bring the multimodal expectation value μ_{nM_j} closer to the target value c_{eT} on the reference axis X₀. The necessary correction Δ_{1_j} of the datum x_{1_j} can be roughly estimated with the control function f₀₁ of the population: Δ_{1_j}≈f₀₁ (c_{eT})−f₀₁ (μ_{nM_j}). This determines the corrected datum x_{1_j+1}:=x_{1_J}+Δ_{1_J} of the status indicator X₁ in iteration stage j+1 (FIG. 10C). The drug-dose-time profile of the infusion pump is adjusted accordingly to achieve the corrected model-based equilibrium state of the variable x_{1_j+1}. In this estimation of the correction value Δ_{1_j}, the new, corrected patient state is \vec{S}ⱼ₊₁ is essentially a parallel shift with respect to the regression function f₀₁. FIGS. 10C and 10D show the result of such a correction with target achievement Δ_{1_j+1}<Δₘᵢₙ. This rough estimation of the correction step may lead to unsatisfactory convergence behaviour in the iteration process in order to approximate the multimodal expectation value μ_{nM} of the target concentration c_{eT}. In order to achieve better convergence behaviour, it is therefore advantageous to perform the correction in several smaller sub-steps:

$${x}_{1 \_ j + 1} = {x}_{1 \_ j} + ε * {Δ}_{1 \_ j} , with ε < 1.$$

As soon as the target value with the condition Δ_{0_j}<Δₘᵢₙ is reached, it is checked at defined time intervals whether the multimodal expectation value is also maintained over time or whether the unknown, true drug concentration, which is approximately described by the multimodal expectation value μ_{nM}, is subject to a drift over time. FIG. 10E shows an example of a drift of the patient state Sⱼ₊₁→Sⱼ₊₂ over time. The iterative correction is shown in FIGS. 10F and 10G. The necessary process steps are shown in the flow diagram in FIG. 9 as control loops 9.6 and 9.7.

PMM-TCI method A uses the regression function f₀₁ of the population as a control function to determine the necessary correction steps of the variable x₁. In the following PMM-TCI methods B, C, D, personalized, multimodal control functions f_{0k_pers} are generated in order to calculate the correction requirement of the x_{1_J} datum for an iterative target approximation of the multimodal expectation value μ_{nM_j} to the defined target value c_{eT}.

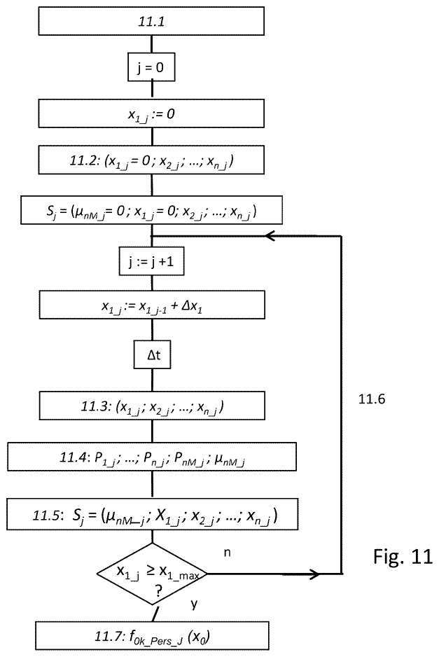

The personalized control functions f_{0k_pers_J} are patient-specific correlation functions between the patient-specific xₖ data of the unimodal state indicators Xₖ and the multimodal expectation values μ_{nM} calculated from them. From the sequence of currently determined patient states \vec{S}ⱼ=(μ_{nM_j}; x_{1S_J}; x_{2_j}; . . . ; x_{n_j}) with j=1; . . . ; J, personalized control functions f_{0k_pers_J} are calculated using a least square distance method in order to approximate a multimodal expectation value μ_{nM} to the target value c_{eT} on the reference axis X₀ with the dependent variable x₁ in an “open-loop” or “closed-loop” control. FIG. 11 describes in a flow chart the process of how the personalized control functions f_{0k_pers} with k=1; . . . ; n are determined. A stress-induced disturbance of the xₖ data is excluded during the generation of the functions f_{0k_pers}.

First, the system configuration is entered in box 11.1, for example the choice of indicators or the target concentrations of the drugs, and in box 11.2 the starting point (zero point) is defined with the measurement by at least one sensor 8.1. \vec{S}₀ is determined. Ideally, the model-based drug concentration x₁ is then increased in several stages of the increment Δx₁ and activated in the infusion pump 8.4. In each stage x_{1_j}, after reaching a stationary equilibrium of the drug concentration in the patient after a period of time Δt, the sensor data x_{k_j} with k=2; . . . ; n are recorded with the sensors 8.1 according to box 11.3 and assigned to the x_{1_j} data. This results in a sequence of patient data {x_{k_j}|k=1; . . . ; n and j=1; . . . ; J}.

For example, the sensors 8.1 provide the data for the state indicators X₂: EEG index and X₃: MAP. This current patient data from box 11.3 is superimposed on the population data stored in the memory of system 8.6. For each stage j, a current, multimodal expectation value μ_{nM_j} of the drug concentration is then calculated in box 11.4 and assigned to the current, unimodal patient data x_{k_j}. This results in box 11.5 in a sequence of patient-specific data points \vec{S}ⱼ=(μ_{nM}; x_{nM_j1_j}; x_{2_j}; . . . ; x) with j=1; . . . ; J in the n+1-dimensional state space by gradually increasing the variable x_{1_J} over the clinically relevant range in the feedback loop 11.6. In field 11.5, the true but unknown drug concentrations x_{0_j} at the time of measurement are substituted by the multimodal expectation values μ_{nM_j}.

FIGS. 12A and 12B show examples of the projection of these patient-specific data points \vec{S}ⱼ into the [X₀; X₁] plane and the [X₀; X₂] plane of the state space. For this sequence of data points, personalized regression functions f_{0k_pers_J}(x₀) in the [X₀; Xₖ] planes of the state space are calculated in box 11.7 using LSD fits. FIGS. 12A and 12B show these personalized regression functions f_{0k_pers_J} (dashed curves) in addition to the regression functions f₀ₖ of the population data (solid curves). For the data point S_{J} projected into the [X₀; X₁] plane, the probability densities P_{k_J} and P_{nM_J} calculated in box 11.4 are also shown.

The personalized regression functions f_{0k_pers_J} describe the patient-individual functional relationship between a unimodal indicator datum x_{k_j} and the multimodal expectation value μ_{nM_j}, which substitutes the true but unknown drug concentration x_{0_j}. These personalized regression functions are used as personalized control functions for “open-loop” or “closed-loop” applications to achieve or maintain a target concentration c_{eT} on the reference axis X₀.

In the following, in particular the control function f_{0k_pers_J}(x₀) shown in FIG. 12 a is used in a PMM-TCI, with which the pharmaco-kinetically calculated drug concentration x_{1_j} is determined as a dependent control variable in order to approximate the multimodal expectation values μ_{nM_j} to the defined target date c_{eT}. The other personalized regression functions f_{0k_pers_J}(x₀) with k=2; . . . ; n are also of inventive, safety-relevant importance in order to prospectively estimate possible violations of boundary conditions of the state indicators Xₖ that could occur during target approach. They can be estimated with x_{k_J+1}:=f_{0k_pers_J}(_{ceT_Prop}) even before the new corrected variable x_{1_J+1} is set and activated.

FIG. 12B shows an example of a limit condition x_{2_min} of the status indicator X₂ that should not be undershot: A target value c_{eT} is defined on the reference axis X₀. The regression function f₀₂ or the personalized regression function f_{02_pers_J} can then also be used to prospectively estimate the expected x₂ datum, x_{2_J+1}≈f₀₂ (c_{eT}) or x_{2_J+1}≈f_{02_pers_J}(c_{eT}), in order to indicate a limit value violation x_{2_J+1}≤ x_{2_min}. For example, the MAP could also fall below a critical value (hypotension) in the event of a planned, corrective increase in the drug concentration x_{1_J+1}.

Such potential patient risks can be avoided by a prospective evaluation of the regression functions f₀ₖ(x₀), in particular with the personalized regression function f_{0k_pers}(x₀) with k=2; . . . ; n even before adjustments of the drug concentration with the controlled variable x_{1_J+1} to be corrected.

The PMM-TCI method B described below uses the previously determined personalized regression functions f_{0k_pers_J} improve the convergence behaviour when approximating the multimodal expectation value to the target value.

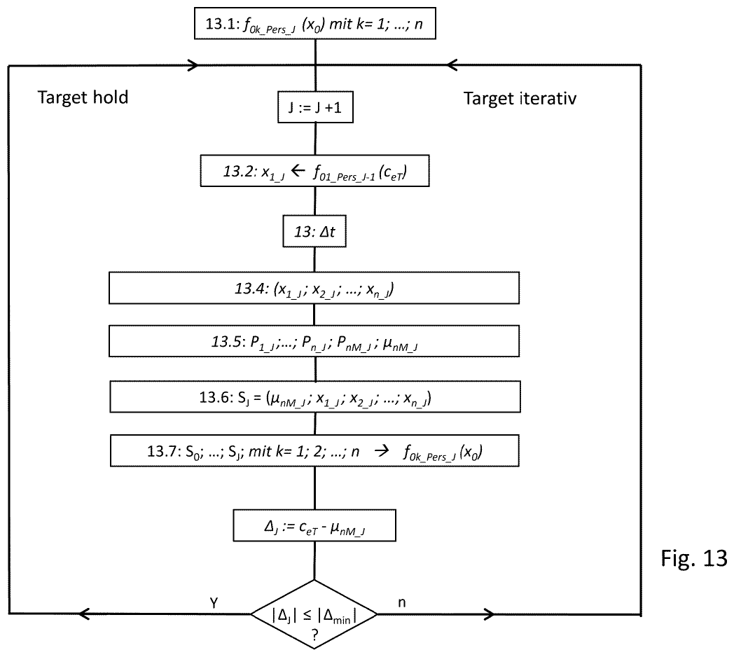

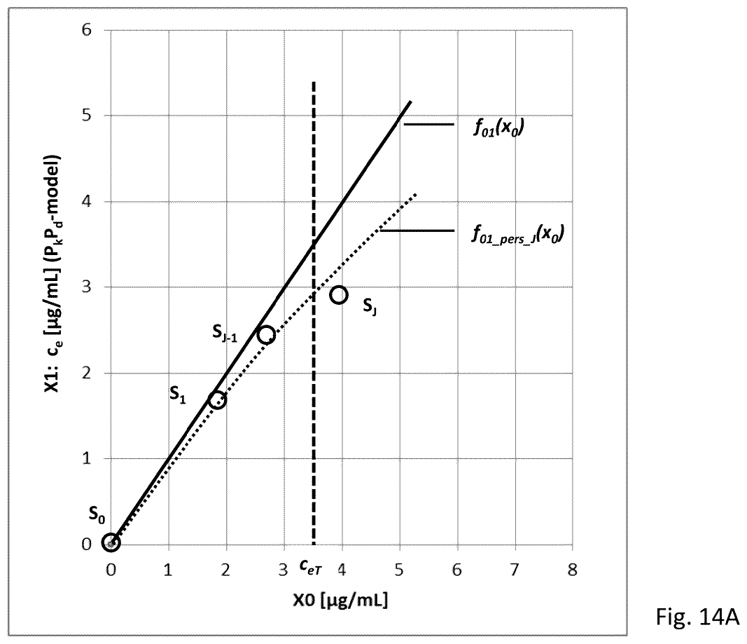

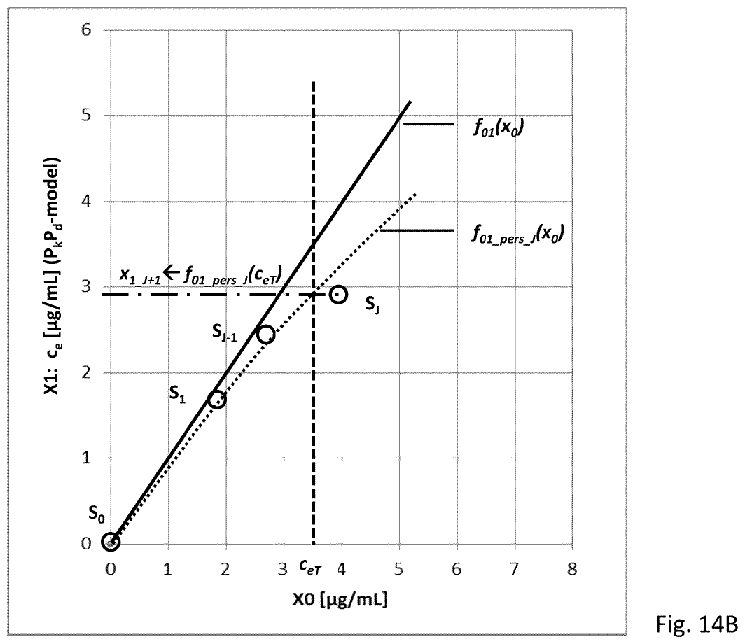

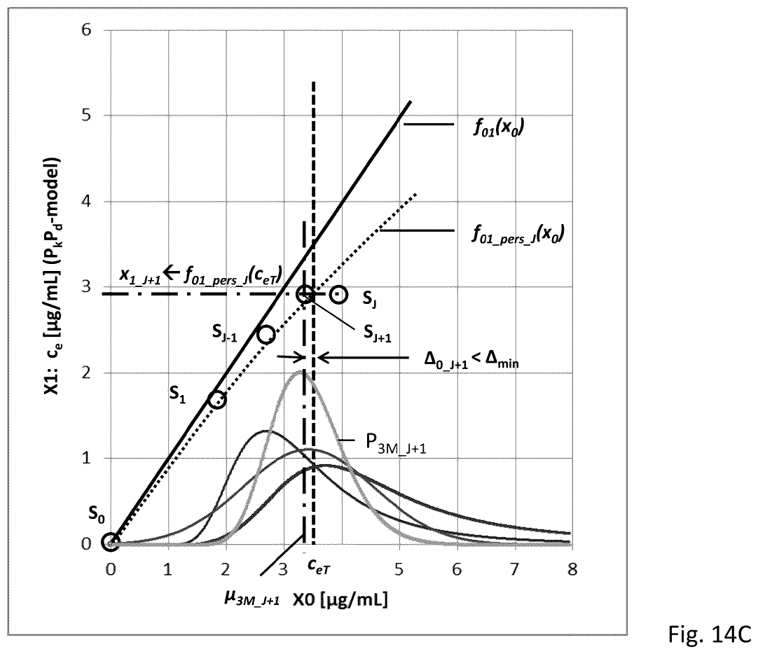

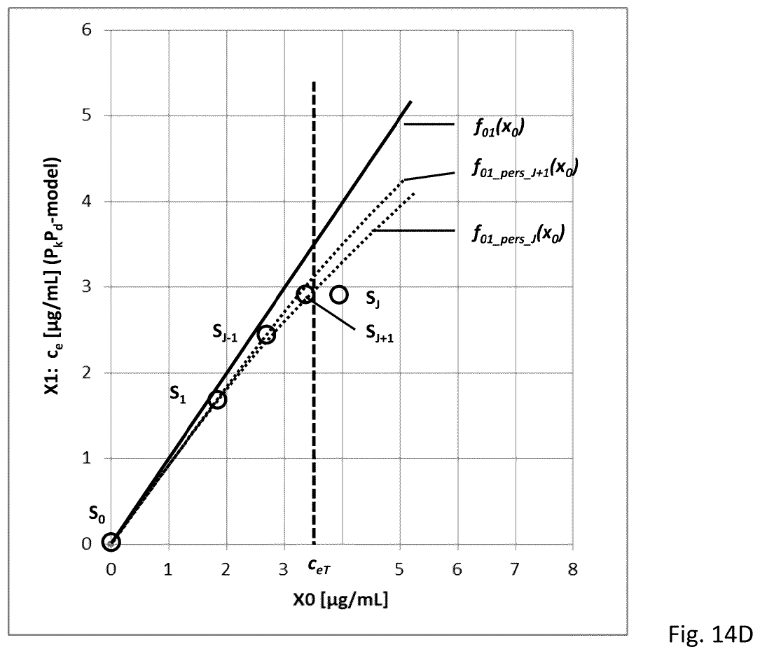

The personalized, multimodal PMM-TCI method B is summarized in the flow chart in FIG. 13 and the individual steps are visualized in FIGS. 14A-C. In particular, method B uses the control function f_{01_pers_J} in the [X₀; X₁] plane of the state space generated in advance according to box 13.1, which was generated without stress-induced disturbance of the xₖ data, in order to approximate the multimodal expectation value μ_{nM_J} to the defined target value c_{eT} on the reference axis X₀ in an iterative “open loop” or “closed loop” control process.

The personalized control function f_{01_pers_J} in the [X₀; X₁] plane and the personalized regression functions f_{0k_pers_J} in the [X₀; Xₖ] planes of the state space were generated with a sequence of patient-individual states \vec{S}ⱼ with j=0; . . . ; J according to the flow chart in FIG. 11. The control function f_{01_pers_J} is used for target achievement. The other personalized regression functions f_{0k_pers_J} are used for the prospective evaluation of possible safety-relevant violations of indicator-specific boundary conditions.

In the following example, it is assumed that this monitoring is active, but that a violation of the limit conditions does not occur. FIG. 14A shows the initial situation in the [X₀; X₁] plane with the regression function of the population f₀₁ and the personalized control function f_{01_pers_J}. A target concentration c_{eT} of the drug is defined on the X₀ axis. In the next step J+1 (according to box 13.2 and FIG. 14B), the dependent variable x_{1_J+1} is calculated, i.e. the model-based drug concentration x_{1_J+1}:=f_{01_pers_J}(c_{eT}), with which the multimodal expectation value μ_{nM} is to be approximated to this target value on the X₀ axis. The x_{1_J+1}—datum is transferred to an established classical PₖP_{d}-TCI module 8.5, with which the infusion pump 8.4 calculates and dynamically activates a suitable drug dose-time profile pharmacokinetically and dynamically in order to set and maintain the transferred drug concentration x_{1_J+1} constant in the model. After a period of time Δt according to box 13.3, a stationary equilibrium of the drug concentration in the patient is reached. The x_{k_J+1} data of the state indicators Xₖ k=2; . . . ; n are then measured with the sensors 8.1 according to box 13.4. According to box 13.5, the multimodal expectation value μ_{nM_J+1} is then calculated and according to box 13.6 the updated state \vec{S}_{J+1}=(μ_{nM_J+1}; x_{1_J+1}; . . . ; x_{n_J+1}) is displayed in the state space. FIG. 14C shows that the updated multimodal expectation value μ_{nM_J+1} already approximately corresponds to the target value c_{eT}.

In FIG. 14D, the updated control function f_{01_pers_J+1}, is calculated in a further iteration step, taking into account the current patient state \vec{S}ⱼ₊₁ according to box 13.7. This updated function is then used to achieve or maintain the target in further iteration steps.

The PMM-TCI method B has an improved convergence behaviour compared to the PMM-TCI method A, since the personalized control function f_{01_pers_J} is used instead of the regression function f₀₁ of the population, and the updated multimodal expectation value μ_{nM_J+1} can therefore be approximated to the target value with fewer iteration steps.

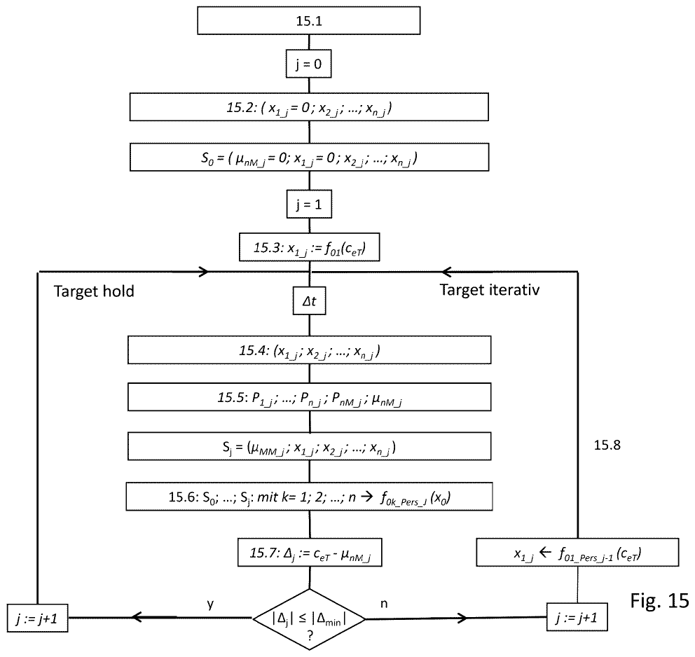

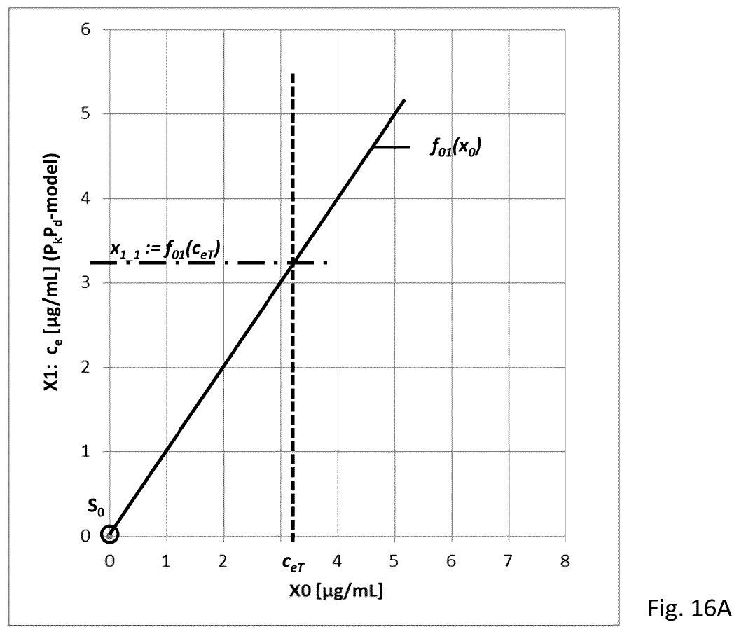

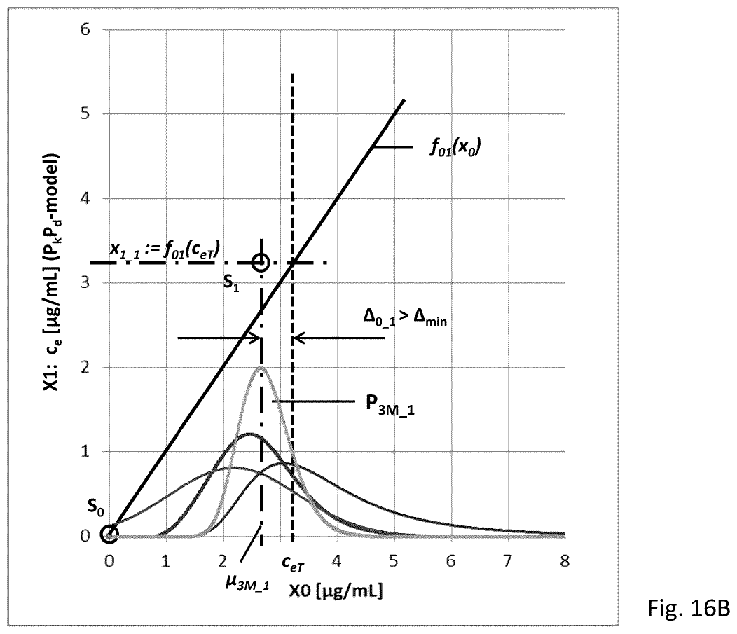

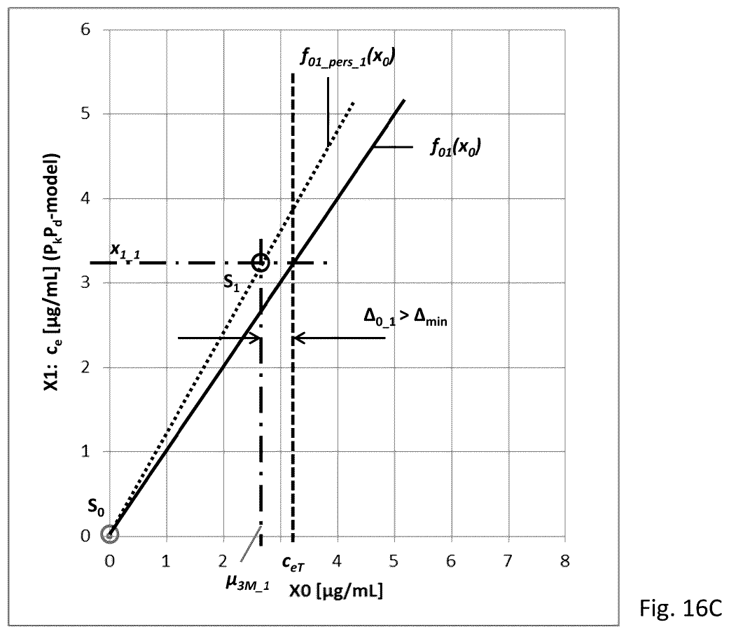

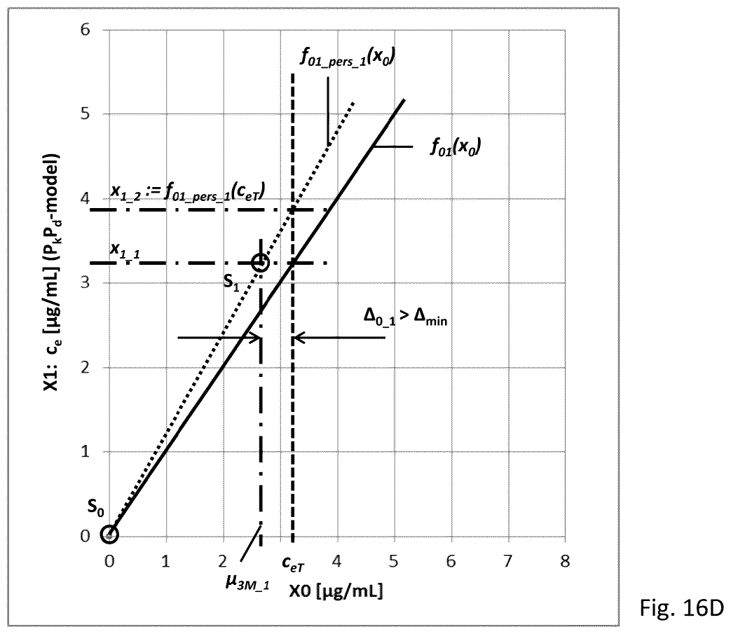

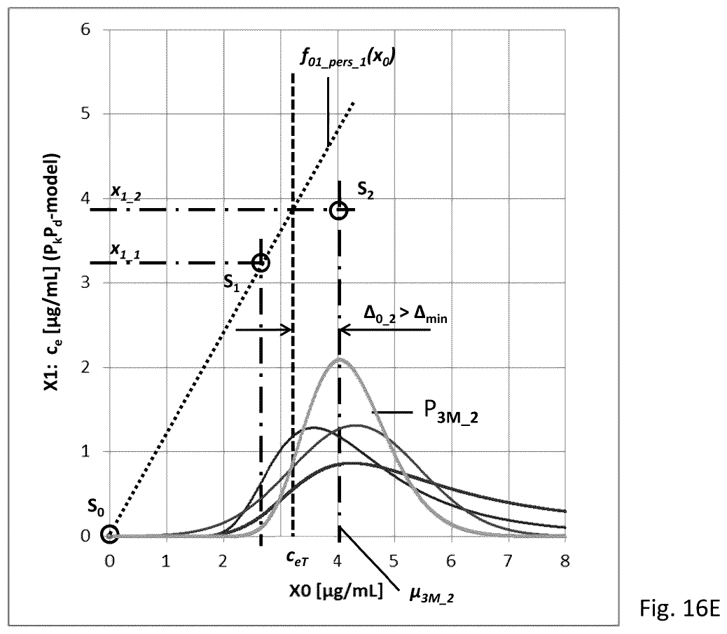

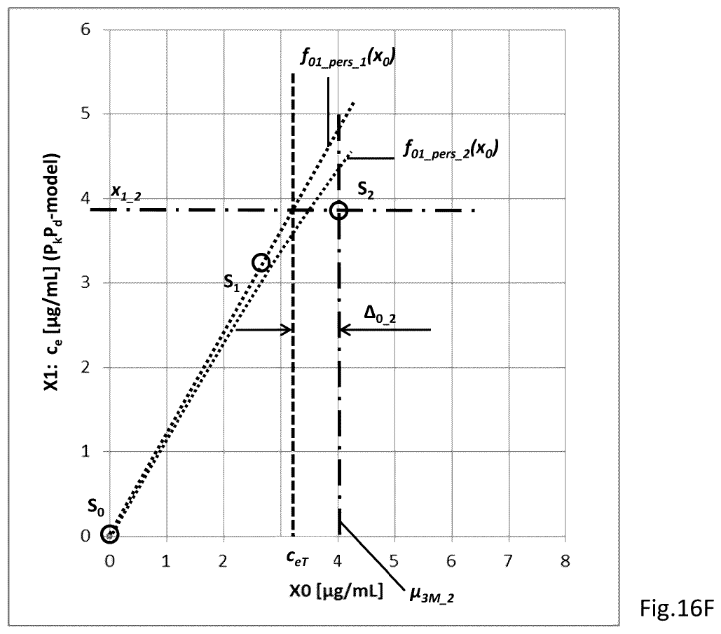

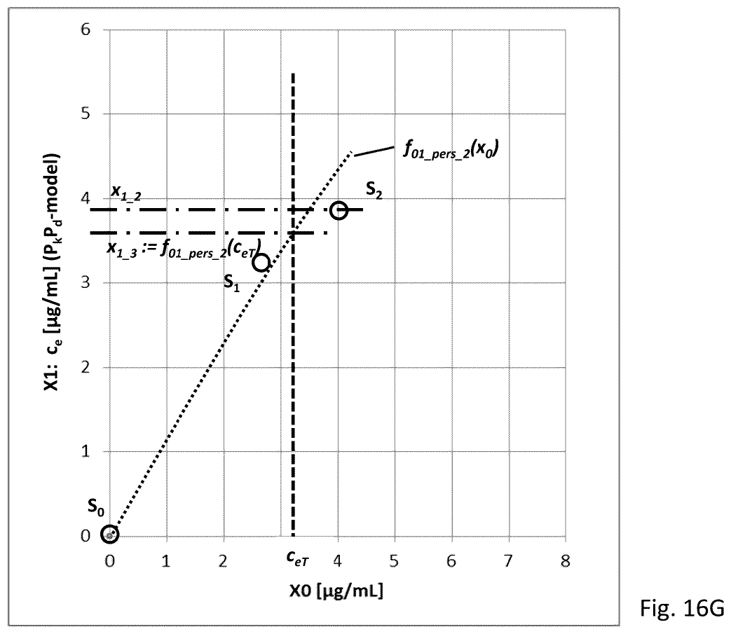

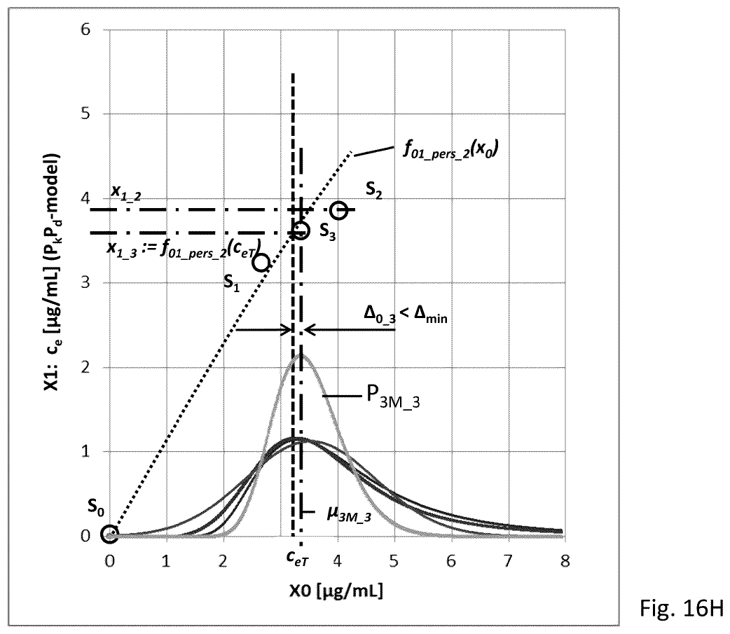

The induction phase is often short in clinical practice, so that personalized regression functions from a sequence of patient data points data points \vec{S}ⱼ=(μ_{nM_j}; x_{1S_j}; x_{2_j}; . . . ; x_{n_j}) with j=1; . . . ; J over the clinically relevant range are not calculated due to time constraints. In order to save time in a PMM-TCI application, it is therefore advantageous, after recording the starting point S to already aim in the first iteration step j=1 a patient state \vec{S}₁=(μ_{nM_1}; x_{1_1})=f₀₁ (c_{eT}; x_{2_1}; . . . ; x_{n_1}) close to the target c_{eT} in order to further approximate the multimodal expectation value μ_{nM_j} to this target c_{eT} on the reference axis X₀ in just a few additional iteration steps j=2; . . . J if necessary. This time-shortened PMM-TCI method C is described in the flow diagram in FIG. 15 and the individual steps are visualized in FIGS. 16A-H.

In box 15.1, a system configuration such as the target value input is carried out, and in box 15.2, the measurement for zero-point determination is carried out with the sensors 8.1 in order to determine the starting point \vec{S}₀ without drug effect. Then, in the first iteration step j=1 according to box 15.3, the variable x_{1_1}:=f₀₁ (c_{eT}) is determined using the regression function f₀₁ of the population and the defined target value c_{eT} (FIG. 16A) and transferred to a classical PₖP_{d}-TCI module 8.5, with which the infusion pump 8.4 calculates and activates a suitable drug dose-time profile pharmacokinetically and dynamically in order to set and maintain a constant drug concentration x_{1_1} in the model. After a time-interval Δt, a stationary equilibrium of the drug concentration in the patient is reached, and the x_{k_1} data of the current unimodal state indicators Xₖ with k=2; . . . ; n of the patient are measured with the sensors 8.1 according to box 15.4. These patient-specific x_{k_1} data are superimposed on the population data in order to calculate the multimodal expectation value μ_{nM_1} and probability densities P_{nM_1} (FIG. 16B) according to box 15.5. In the example shown, the difference Δ_{0_1}>Δₘᵢₙ, and μ_{nM_1} is close to the defined target value c_{eT}, but a further correction is required to bring the patient condition closer to the target. First, the already recorded patient-individual conditions \vec{S}₀ and \vec{S}₁ are used to calculate personalized regression functions f_{0k_pers_1} in box 15.6. For this purpose, the coefficients of the regression functions f₀ₖ (x₀) based on population data are adjusted to the sequence of current patient data points S₀ and S₁ in the respective [X₀; Xₖ] planes of the state space using a linear or non-linear least square distance method, for example the Gauss-Newton method. The result of this adaptation are personalized regression functions f_{0k_pers_1}(x₀) with k=1; . . . ; n of iteration level j=1. FIG. 16C shows the result in the [X₀; X₁] plane of the state space. After the first iteration, it can be assumed that the personalized regression functions f_{0k_pers_1} due to the small number of patient states \vec{S}ⱼ with j=0, 1 still have a low accuracy over the entire clinically relevant range. In the local environment of the target value c_{eT}, iteration level 1 should already be suitable for better approximating the multimodal expectation value μ_{nM_1} to the target value c_{eT} according to box 15.7. If necessary, further iteration steps follow: In the next iteration step j=2, a corrected variable x_{1_2}:=f_{1_pers_1}(c_{eT}) is determined in the feedback loop 15.8 (FIG. 16D). Again, the corrected dependent variable x_{1_2} is passed to the classical PₖP_{d}-TCI module 8.5 to calculate a suitable drug dose-time profile pharmacokinetically and dynamically. The infusion pump 8.4 activates this profile to reach the corrected date x_{1_2} and maintain it constant in this model. The corrected patient condition \vec{S}₂ is then determined at the steady-state equilibrium (FIG. 16E). In the example shown, the patient state S₂ in the [X₀; X₁] plane is not on the correlation function f_{01_pers_1}, but clearly off it: Δ_{0_2}>Δₘᵢₙ. The correction of the deviation Δ_{0_1} was obviously overcompensated in the example. The new, current state S₂ is then used to calculate corrected regression functions f_{0k_per_2} of iteration step j=2 in accordance with box 15.6 (FIG. 16F) in order to determine a new correction value x_{1_3} (FIG. 16G). If necessary, further iteration steps j with updated regression functions f_{0k_pers_j} are required (FIG. 16H) until the target achievement is confirmed with Δ_{0_J}≤Δₘᵢₙ.

With the PMM-TCI of method C, the projections of the generated patient states \vec{S}ⱼ with j=1; . . . ; J are grouped around the target point c_{eT} on the reference axis. The regression functions f_{0k_pers_J} are therefore well defined locally around the target value c_{eT}, but not far outside this target range. However, the local accuracy is sufficient for the iterative target adjustment of the expectation value μ_{nM_j} to the target value.

It should also be noted here that the overcompensation shown in the example when approximating the multimodal expectation value μ_{nM_j} to the target value c_{eT} could be avoided by a reduced correction step size ε*(x_{1_j}−x_{1_j+1}) with ε<1. This possibility is obvious and is therefore not discussed further.

It can also be advantageous to weight patient conditions \vec{S}ⱼ with regard to the time of their occurrence. The more recent the condition is, the greater its weighting. In reality, it is generally the case that the true, unknown drug concentration x_{0_j} does not remain constant over time and then causes a drift in the dependent x_{k_j} data. The temporal weighting of the \vec{S}ⱼ-patient states take this possible drift into account and updates the personalized regression functions f_{0k_pers_J} according to the drift behaviour, whereby older states are weighted lower in order to obtain updated, personalized regression functions of the \vec{S}ⱼ-patient states using least square distance methods.

The PMM-TCI method D corrects the target concentration of the anaesthetic drug, for example Propofol c_{eT_Prop}, and also the target concentration of the analgesic drug, for example Remifentanil c_{eT_Remi}, in an open-loop or closed-loop application in order to minimize patient reactions due to stress induction during a surgical procedure. For this purpose, it is assumed that a population data set exists for the anaesthetic drug, for example POP-Propofol, which contains the xₖ data of at least two unimodal state indicators X_{k_Prop} and a reference indicator X_{0_Prop}, which are indicative of the depth of anaesthesia. In addition, there is a population data set for the analgesic drug, for example POP-Remifentanil, with at least one unimodal state indicator X_{1_Remi} and one reference indicator X_{0_Remi}, which are indicative of analgesia. In the following detailed description of the PMM-TCI method D, it is assumed for simplicity without limiting generality that the POP-Remi dataset consists only of the data of the state indicator X_{1_Remi} and the reference Indicator X_{0_Remi}, and cross-correlations of the effect of the two drugs on the state indicators are neglectable. The data sets are mapped in a common population in the extended state space.

In the induction phase, a phase without any stress effect on the patient, the multimodal expectation value μ_{nM_j} of the target concentration for propofol c_{eT_Prop} is approximated and maintained using the PMM-TCI methods A, B or C. In this phase, the target concentration c_{eT} Ren of Remifentanil is also set and achieved in the model using an established PₖP_{d}-TCI algorithm, for example a Minto model.

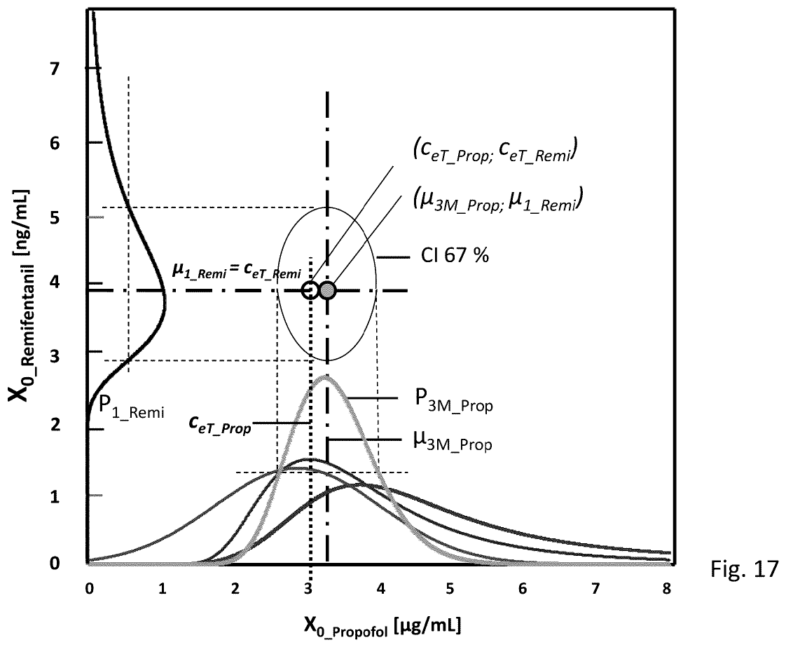

FIG. 17 visualizes an example of the [X_{0_Prop}; X_{0_Remi}] plane of the extended state space with two reference indicators X_{0_Prop} and X_{0_Remi}, which is displayed on the user interface 8.7 of the system, and in which the target achievement and maintenance of the anaesthesia and analgesia state can be monitored by the user in an “open loop” or “closed loop” control. The probability densities of a measurement with three unimodal state indicators and the triple-modal expectation value μ_{3M_Prop}≈3.25 μg/mL as well as the corresponding triple-modal probability density P_{3M_Prop} are plotted along the reference axis X_{0_Prop}. With μ_{3M_Prop}≈c_{eT_Prop}=3.0 μg/mL, the defined target concentration for propofol is approximately reached. The unimodal expectation value μ_{1_Remi}, the corresponding probability density P_{1_Remi} and the target concentration c_{eT_Remi} are plotted along the reference axis X_{0_Remi}. With μ_{1_Remi}=c_{eT_Remi}=3.9 ng/mL, the defined target concentration for Remifentanil is also reached. The coordinate point (μ_{3M_Prop}; μ_{1_Remi}) is the visualized expectation state in the [X_{0_Prop}; X_{0_Remi}] plane of the state space, which essentially coincides with the target point (c_{eT_Prop}; c_{eT_Remi}). The triple-modal probability density P_{3M_Prop} provides a narrow, personalized confidence interval for the expectation value μ_{3M_Prop} on the reference axis X_{0_Prop}. On the X_{0_Remi} axis, the unimodal probability density P_{1_Remi} provides a wider confidence interval for the unimodal expectation value μ_{1_Remi}. Together, this results in an adaptive, elliptical confidence interval in the plane assigned to the coordinate point (μ_{3M_Prop}; μ_{1_Remi}). In the example shown, the multimodal expectation value μ_{3M_Prop}, can be controlled according to the PMM-TCI methods A, B, C in a “closed-loop” or a manual “open-loop” procedure in order to obtain the defined target value c_{eT_Prop}. Control according to the PMM-TCI methods A, B, C would also be feasible in principle for the Remifentanil expectation value along the X_{0_Remi} axis if, in addition to the state indicator X_{1_Remi}, at least one remifentanil-specific unimodal state indicator X_{k_Remi} referencing physiological data were used to calculate personalized, multimodal expectation values μ_{nM_Remi}. However, this is not discussed in detail.

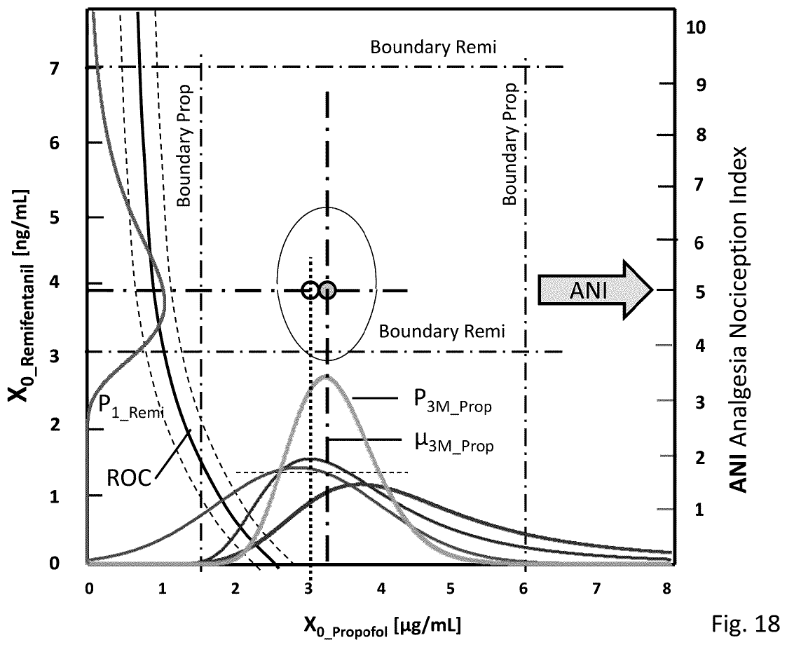

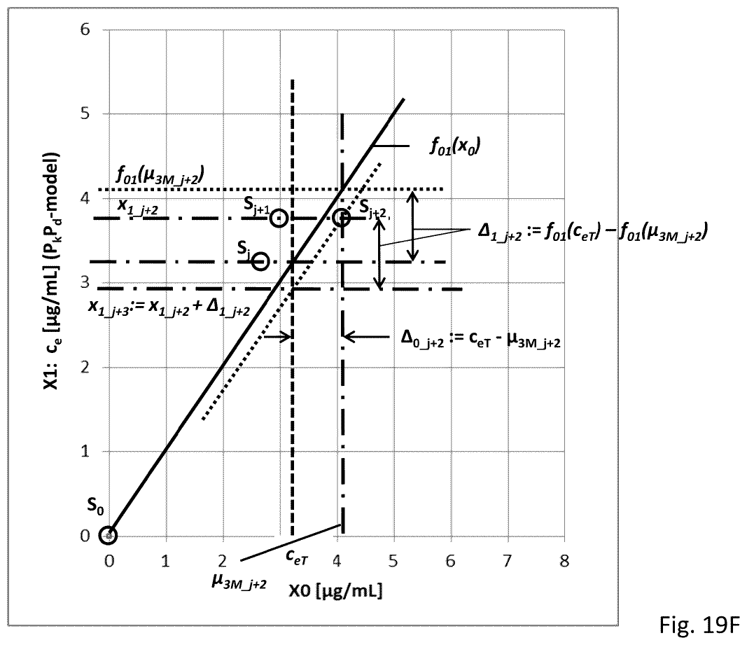

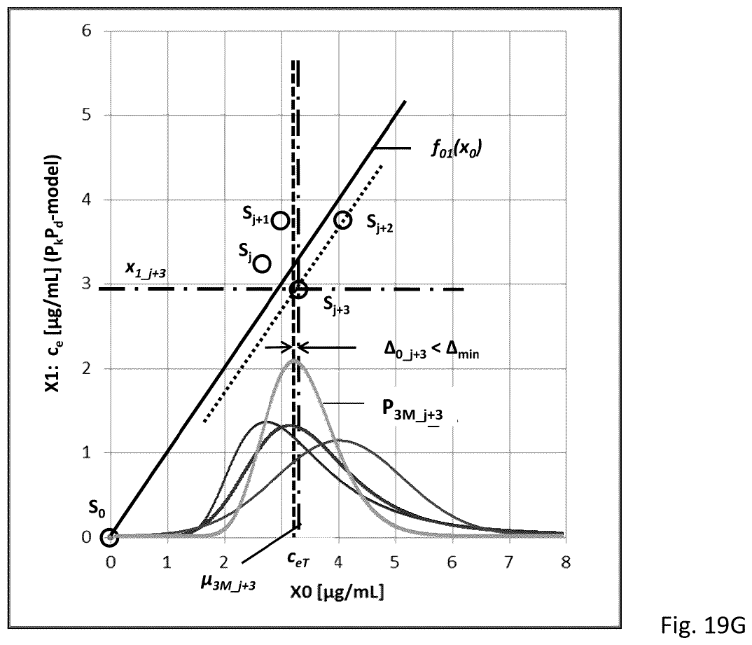

It is advantageous if, in addition to the target point (c_{eT_Prop}; c_{eT_Remi}) and the expected state (μ_{nM_Prop}, μ_{1_Remi}), boundary conditions of clinical relevance are also defined in the [X_{0_Prop}; X_{0_Remi}] plane of the state space visualized on the monitor 8.7. FIG. 18 shows an example of the ROC landmark for “Return of Consciousness”. The LOI landmark—“Loss of Consciousness”—would also be an important boundary condition. The LOI data and the ROI data are stored in memory 8.6 in the population data set. The ROC curve shows an example of the additive effect of the anaesthetic and the analgesic drugs over the clinically relevant range. The representation of the distance of the target state and the expectation state from these landmarks and their associated confidence intervals is helpful as risk-minimizing information. In addition, upper and lower limits can be set which should not be exceeded or undershot in open-loop or closed-loop procedures. An alarm is displayed if a limit violation is expected.

ANI index scales (analgesia-nociception index) (10) are also known, which quantify the current, induced stress due to the surgical procedure on the basis of physiological data. They are based, for example, on hemodynamic measurement data such as heart rate (HR) or heart rate variability (HRV). The hemodynamic measurement data can also be displayed directly on a scale of the monitor 8.7.

In FIG. 18, a ANI index scale is visualized as an example on the right-hand side of the plot. If defined limit values for the induced stress with regard to the HR data or the HRV data or the ANI index values are violated, or if it is foreseeable that they will be violated, the target state (c_{eT_Prop}; c_{eT_Remi}) can be shifted. The direction of the shift in the [X_{0_Prop}; X_{0_Remi}] plane must be selected in such a way that expected limit violations are avoided. The expectation state (μ_{3M_Prop}, μ_{1_Remi}) is then approximated to the shifted target state using the specific PMM-TCI-Prop methods for the anaesthetic drug and/or TCI-Remi algorithms for the analgesic drug. Manual “open-loop” or automatic “closed-loop” methods, or a combination of both methods, can be used for this purpose.

1-40. (canceled)

41. Method for determining a multimodal (patient) condition, characterized in that
a) a current (patient) state consisting of data from at least two unimodal state indicators Xₖ with k=1; . . . ; n and n≥2 is measured or calculated;
b) a historical (population) data set with data from at least two state indicators Xₖ and at least one reference indicator X₀ exists;
c) there is a correlation between the at least two state indicators on the one hand and the at least one reference indicator on the other;
d) the at least two state indicators Xₖ and the at least one reference indicator X₀ span an orthogonal state space;
e) Regression functions can be calculated in the state space with the historical (population) data set, which contain at least one reference indicator X₀ as an independent variable;
f) and at least one current, n-modal (n≥2) expectation value μ_{nM} of the currently unknown, effective reference value on at least one reference axis X₀ of the state space is calculated for a current (patient) state using the regression functions.

42. Method according to claim 41, characterized in that for a current (patient) state, consisting of data from at least two unimodal state indicators Xₖ, an n-modal expectation value μ_{nM} is calculated by averaging the unimodal expectation values μₖ of the currently unknown (patient) reference value x₀ on the reference axis X₀.

43. Method according to claim 41, characterized in that for a current (patient) state, consisting of data from at least two unimodal state indicators Xₖ, current unimodal probability densities P₀ₖ of the currently unknown (patient) reference value x₀ are calculated and a multimodal probability density μ_{nM} and an n-modal expectation value μ_{nM} are calculated by convolution on the reference axis X₀.

44. Method according to claim 41, characterized in that at least one unimodal state indicator Xₖ is derived from physiological measurement data and at least one unimodal state indicator Xₚ is calculated from pharmaco-kinetic-dynamically calculated model data of a drug concentration in the blood plasma or at effect site of test subjects or patients.

45. Method according to claim 41, characterized in that a sequence of current data {x_{k_j}|k=1; . . . ; n; j=1; 2; . . . ; J) of the state indicators Xₖ and the sequence of multimodal expectation values (μ_{nM_j}|j=1; 2; . . . ; J} is used to generate a sequence of states {(μ_{nM_j}; x_{k_j})|k=1; . . . ; n; j=1; 2; . . . ; J} in the [X₀; Xₖ] planes, for which personalized regression functions f_{0k_pers_J}(x₀) are calculated using least square distance fit methods.

46. Method according to claim 41, characterized in that the sequence of data {(μ_{nM_j}; x_{k_j})|k=1; . . . ; n; j=1; 2; . . . ; J} are weighted with respect to their time of origin.

47. Method according to claim 41, characterized in that at least one target concentration c_{T} of a drug is defined on at least one reference axis X₀ of the state space and the target deviation Δ=μ_{nM}−c_{T} of a current multimodal expectation value μ_{nM} from the target value c_{T} is determined on at least one reference axis X₀ of the state space.

48. Method according to claim 41, characterized in that at least one pharmaco-kinetically, -dynamically calculated drug concentration x₁ of a state indicator X₁ is used as a dependent control variable, with which the target deviation Δ=μ_{nM}−c_{T} is minimized.

49. Method according to claim 41, characterized in that the regression function f₀₁(x₀) or the personalized regression function f_{01_pers_J}(x₀) in the [X₀; X₁]plane of the state space is used as a control function with which the target deviation Δ=μ_{nM}−c_{T} is minimized.

50. Method according to claim 41, characterized in that the iterative process for achieving and maintaining a minimal target deviation Δ=μ_{nM}−c_{T} is implemented as an ‘open-loop’ application or as a ‘closed-loop’ application

51. Method according to claim 41, characterized in that the regression functions f₀ₖ of the population or the personalized multimodal regression functions f_{0k_pers_J} in the [X₀; Xₖ] planes of the state space are used to prospectively calculate the data f₀ₖ (c_{T}) or f_{0k_pers_J}(C_{T}) of the state indicators Xₖ with k=1; . . . ; n, which are to be expected when the target is reached, in order to indicate and avoid possible borderline violations of these data before the target value c_{T} is reached.

52. Method according to claim 41, characterized in that the target concentration c_{T} and the multimodal expectation value μ_{nM} or the unimodal expectation values μₖ of the state indicators Xₖ are mapped on at least one reference axis X₀ and visualized with a user interface.

53. Method according to claim 41, characterized in that the expectation values visualized on at least one reference axis X₀ are also assigned to the probability densities or confidence intervals CI derived therefrom and displayed in a user interface.

54. Method according to claim 41, characterized in that a trend development is calculated from the temporal sequence of the multimodal patient states in the [X₀; Xₖ] planes of the state space and visualized with a user interface.

55. Method according to claim 41, characterized in that for two drugs A and B in a plane which is spanned by the orthogonal reference axes X_{0_A} and X_{0_B}, the target state (c_{T_A}; c_{T_B}) and the multimodal expectation state (μ_{nM_A}; μ_{pM_B}) are visualized with a user interface, where μ_{nM_A} is the n-modal expectation value with n≥2, μ_{pM_B} is the p-modal expectation value with p≥1 and c_{T_A} and c_{T_B} are the target values of drugs A and B on their respective reference axes X_{0_A} and X_{0_B}.

56. Device for determining a multimodal (patient) condition, characterized in that
a) a current (patient) state consisting of data from at least two unimodal state indicators Xₖ with k=1; . . . ; n and n≥2 can be measured or calculated;
b) a historical (population) data set with data from at least two state indicators Xₖ and at least one reference indicator X₀ exists;
c) there is a correlation between the at least two state indicators on the one hand and the at least one reference indicator on the other;
d) the at least two state indicators Xₖ and the at least one reference indicator X₀ span an orthogonal state space;
e) Regression functions can be calculated in the state space with the historical (population) data set that contain at least one reference indicator X₀ as an independent variable;
f) and at least one current, n-modal (n≥2) expectation value μ_{nM} of the currently unknown, effective reference value on at least one reference axis X₀ of the state space can be calculated for a current (patient) state using the regression functions.

57. Device according to claim 56, characterized in that for a current (patient) state, consisting of data from at least two unimodal state indicators Xₖ, an n-modal expectation value μ_{nM} is calculated by averaging the unimodal expectation values μₖ of the currently unknown (patient) reference value x₀ on the reference axis X₀.

58. Device according to claim 56, characterized in that for a current (patient) state, consisting of data from at least two unimodal state indicators Xₖ, current unimodal probability densities P₀ₖ of the currently unknown (patient) reference value x₀ are determined and a multimodal probability density P_{nM} and an n-modal expectation value μ_{nM} are calculated by convolution on the reference axis X₀.

59. Device according to claim 56, characterized in that at least one unimodal state indicator Xₖ is derived from physiological measurement data and at least one unimodal state indicator Xₖ is calculated from pharmaco-kinetically-dynamically calculated model data of a drug concentration in the blood plasma or at effect site of test subjects or patients.

60. Device according to claim 56, characterized in that for two drugs A and B in a plane which is spanned by the orthogonal reference axes X_{0_A} and X_{0_B}, the target state (c_{T_A}; c_{T_B}) and the multimodal expectation state (μ_{nM_A}; μ_{pM_B}) are visualized with a user interface, where μ_{nM_A} is the n-modal expectation value of drug A with n≥2, μ_{pM_B} is the p-modal expectation value pf drug B with p≥1 and c_{T_A} and c_{T_B} are the target values of drugs A and B on their respective reference axes X_{0_A} and X_{0_B}.

61. Device according to claim 56, characterized in that a control unit with a data memory and a data processing device is provided, as well as an input interface for receiving unimodal patient data, and an output interface for controlling an infusion device for administering at least one anaesthetic drug to a patient, wherein the infusion device can calculate a quantity of the anaesthetic drug in accordance with the patient data received and the information stored in the data memory, such as in particular the historical data record, in order to be able to place the patient in a sufficiently deep anaesthesia, and wherein the infusion device can be controlled via the output interface to deliver the corresponding quantity of the anaesthetic drugs in terms of duration and quantity.
